(19) United States
(12) Patent Application Publication
(10) Pub. No.: US 2009/0076111 A1
(43) Pub. Date: Mar. 19, 2009

(54) Methods for improving glycemic control in humans

(76) Inventors: Steve S. Lee, Sandy, UT (US); Richard B. Hynson, Missoula, MT (US); Ke-Qin Zhang, Fuzhou (CN); Wu-Zhou Li, Xuzhou (CN); Jing Shi Zhou, Shanghai (CN)

Correspondence Address: PATE PIERCE & BAIRD, 175 SOUTH MAIN STREET, SUITE 1250, SALT LAKE CITY, UT, 84111, US

(21) Appl. No.: 11/975,192

(22) Filed: Oct. 18, 2007

Related U.S. Application Data
(63) Continuation-in-part of application No. 11/068,733, filed on Mar. 1, 2005, now abandoned.
(60) Provisional application No. 60/549,198, filed on Mar. 2, 2004.

(51) Int. Cl. A61K 31/197; A61P 3/00; A61K 31/4172

(52) U.S. Cl. 514/400; 514/561; 514/562

(57) ABSTRACT

The present invention is directed to methods for improving glycemic control in humans and animals comprising the step of administering a composition comprising an amino acid content including 4-hydroxyisoleucine in an amount between about 60% and about 70% of a total weight of the amino acid content, together with one or more amino acids selected from the group consisting of glutamate, aspartate, arginine, cysteine, threonine, serine, glycine, alanine, valine, methionine, isoleucine, and histidine (inclusive of any chemical salts, anhydrides, or isomers of any of the foregoing), in addition to, alkaloids, glycosides, volatile oils, saponins, sapogenins, mannans, flavonoids, fatty acids, vitamins and provitamins, minerals, and carbohydrates. Fasting blood glucose and glucose tolerance were studied in normal human subjects, in human subjects diagnosed with Metabolic Syndrome X, and in diabetic rats by means of dosing the subjects with compositions comprising 4-hydroxyisoleucine in an amount between about 20% and about 30% of the total weight of the composition, wherein improving glycemic control in standard glucose tolerance tests.

### RELATED APPLICATIONS

This application claims the benefit of U.S. Provisional Patent Application Ser. No. 60/549,198, filed Mar. 2, 2004, and entitled “AMINO ACID COMPOSITIONS DERIVED, ISOLATED, AND/OR EXTRACTED FROM FENUGREEK SEED,” and co-pending U.S. patent application Ser. No. 10/434,444, filed May 7, 2003, and entitled “FENUGREEK SEED BIO-ACTIVE COMPOSITIONS AND METHODS FOR EXTRACTING SAME,” which claims priority to U.S. Provisional Application Ser. No. 60/379,839, filed May 10, 2002, and entitled “BIO-ACTIVE FENUGREEK SEED COMPONENT EXTRACTION METHOD,” and the benefit of co-pending U.S. patent application Ser. No. 10/926,849, filed Aug. 26, 2004, and entitled “COMPOSITIONS AND METHODS FOR GLYCOGEN SYNTHESIS,” which claims priority to U.S. Provisional Application Ser. No. 60/498,717, filed Aug. 28, 2003, and entitled “COMPOSITIONS AND METHODS FOR GLYCOGEN SYNTHESIS, and the benefit of co-pending U.S. patent application Ser. No. 11/068,734, filed Mar. 1, 2005, and entitled “METHODS FOR ENHANCING THE TRANSPORT OF GLUCOSE FOR BALANCING BLOOD SUGAR LEVELS,” and the benefit of co-pending U.S. patent application Ser. No. 11/068,733, filed Mar. 1, 2005, and entitled “METHODS FOR ENHANCING THE TRANSPORT OF GLUCOSE INTO MUSCLE,” all of which are incorporated herein by reference.

### BACKGROUND

1. Field of the Invention

This invention relates to compositions and methods for extracting and separating bio-active compounds and, more particularly, to novel compositions of bio-active compounds which may be derived, isolated, and/or extracted from Fenugreek seeds and methods for using said compositions for improving glycemic control in humans.

2. The Background Art

Fenugreek is one of the oldest medicinal herbs and has been found to be native to southeastern Europe, northern Africa, and western Asia, although it is widely cultivated in other parts of the world. Fenugreek is known technically as Trigonella foenum-graecum, a member of the family Fabaceae, and commonly referred to as Greek hay.

As appreciated by those skilled in the art, fenugreek is a legume and typically grows between two to three feet tall with light green leaves and small white flowers. A fenugreek seed pod may contain between ten to twenty small, flat, yellowish-brown seeds. Typically, a plant seed is formed having a thick or hard outer coat called a testa and often referred to as a seed coat. The inner portion of the seed coat usually contains a plant embryo and a nutritive tissue called endosperm, which surrounds the embryo. As the fenugreek seed embryo matures, it consumes the endosperm. As appreciated, fenugreek seeds often have a pungent aroma and may have a bitter taste, which is said to be similar to celery.

Fenugreek has long been used as a medicinal herb and culinary additive in both Asia and the Mediterranean. As used herein, a “bio-active compound” may be defined as any substance that has an effect on living tissue. It is believed that the seed of the fenugreek plant contains many active compounds with pharmaceutical applications such as, for example, iron, vitamin A, vitamin B, vitamin C, phosphates, flavonoids, saponins, trigonelline, and other alkaloids. Fenugreek may be taken as a stomach tonic and as a treatment for abdominal ailments. Western scientific research has provided insight into the chemical analysis of fenugreek seeds, together with the extraction of 4-hydroxyisoleucine from fenugreek seeds, and has, accordingly, suggested some clinical utilities of fenugreek.

Early research into the analysis of fenugreek by Sir L. Fowden taught the isolation and purification of 4-hydroxyisoleucine (also referred to hereinafter as 4-OH-Ile) from fenugreek. Fowden claimed that 4-OH-Ile is the principal unbound amino acid contained in fenugreek seed. In addition to 4-OH-Ile, Fowden found that fenugreek also contains gamma-aminobutyrate, ammonia, lysine, histidine, arginine, and at least four (4) additional unidentified compounds. (See, Fowden et al, Phytochemistry, 12:1707, (1973).) Further investigation of the prior art suggests that the amino acids found in fenugreek seeds may have some nutritional value. (See, Sauvaire et al, Nutr Rep Int, 14:527 (1976).)

Spectrophotometry methods have also been taught by those skilled in the art in the analysis of steroid sapogenin content of fenugreek seeds and such prior art methods may be generally used in an effort to determine the composition of subfractions of defatted fenugreek. (See, Baccou et al, Analyst, 102:458 (1977); Ribes et al, Proc Soc Exp Biol Med, 182:159 (1986).) In addition, those skilled in the art have used chloroform in an effort to extract 4-OH-Ile from fenugreek seeds. (See, Alcock et al, Phytochemistry, 28(7):1835 (1989).) It has been found, however, that chloroform is toxic and generally unacceptable as an extraction method under standards established by the food and drug industry.

Studies have also shown that the natural analogue of 4-OH-Ile is more effective as an antidiabetic agent than a synthetic version. There is, therefore, a suggestion that the therapeutic effects of 4-OH-Ile are best obtained from extracts of the fenugreek seed. However, using fenugreek seeds as a raw material source for a nutritional supplement presents some difficulties. For example, one such difficulty or disadvantage stems from the fact that a large dose of fenugreek seeds is usually needed in order to obtain therapeutic and other nutritional effects. Patients or consumers are often unwilling to incorporate even de-fatted and de-bitterized seeds into their diet. As readily appreciated, mild gastro-intestinal upset may occur at higher doses with non-defatted seeds. In addition, due to the high fiber content of fenugreek seeds, prolonged and high dosage amounts may result in adverse side effects such as flatus or diarrhea.

During the research investigations described herein, those skilled in the art developed crude methods for extracting 4-OH-Ile from fenugreek seeds. These prior art methods and extraction techniques have primarily focused on obtaining a “high-purity” extract of 4-OH-Ile. For example, the extraction of 4-OH-Ile using adsorption chromatography is known in the art. Such prior art methods, however, tend to yield only small quantities of 4-OH-Ile and are typically only suitable for small scale laboratory use.

As indicated above, other compounds have also been isolated from fenugreek seeds. In addition to the major isomers (2S,3R,4S)-4-hydroxyisoleucine, minor isomers of 4-OH-Ile, and amino acids (including, lysine, histidine, and arginine) have been isolated. Later studies confirmed the presence of 4-OH-Ile in fenugreek seeds in two diastereoisomers: the major one being the (2S,3R,4S) configuration, representing about ninety percent (90%) of the total content of 4-OH-Ile, and the minor one being the (2R,3R,4S) configuration. (See, Alcock, Phytochemistry, 28:1835 (1989).)

As appreciated by those skilled in the art, the major isomer (2S,3R,4S) is presently interesting with respect to experimental evidence indicating its ability to stimulate glucose-induced insulin secretion in micromolar concentrations through a direct effect on pancreatic beta cell stimulation in a glucose dependent manner. Moreover, 4-OH-Ile is able to interact and induce additive insulinotropic effects (i.e., stimulating or affecting the production and activity of insulin, only in the presence of supranormal glucose concentrations). (See, Sauvaire et al, Diabetes, 47:206 (1998).)

Investigation of the prior art also discloses clinical studies to investigate the use of subfractions of fenugreek in conditions of hyperglycemia, glucosuria, and hyperlipidemia which have been performed on rats, dogs, and human pancreatic tissue. (See, Shani et al, Arch Intern Pharmacodyn Ther, 210:27 (1974); Ribes et al, Ann Nutr Metab, 28: 37 (1984); Valette et al, Athersclerosis, 50:105 (1984); Madar, Nutr Rep Int, 29:1267 (1984).)

Clinical studies directed to conditions of hyperglycemia, as well as other conditions involving the metabolism of carbohydrates, have only investigated 4-OH-Ile as an effector of insulin-mediated or insulin-dependent pathways. The available prior art does not teach or suggest, however, fenugreek and/or 4-OH-Ile compositions which may work synergistically or independently from insulin or insulin-mediated pathways. More particularly, there are no known prior art teachings or suggestions that 4-OH-Ile may affect the body by an insulin-independent mechanism. Stimulation of non-insulin mediated pathways may be desirous for targeting the utilization of carbohydrates in certain organ systems, (e.g., muscles, liver, etc.). Likewise, it may be desirable to avoid the general and/or systemic effects that may occur by stimulating the pancreas to produce and secrete insulin.

As appreciated by those skilled in the art, the major isomer of 4-OH-Ile (2S,3R,4S) is presently interesting with respect to experimental evidence indicating its ability to stimulate glucose-induced insulin secretion in micromolar concentrations through a direct effect on pancreatic beta cell stimulation in a glucose dependent manner. Moreover, 4-OH-Ile is able to interact and induce additive insulinotropic effects (i.e., stimulating or affecting the production and activity of insulin, only in the presence of supranormal glucose concentrations). (See, Sauvaire et al, Diabetes, 47:206 (1998).)

As further appreciated by those skilled in the art, clinical studies directed to conditions of hyperglycemia, as well as other conditions involving abnormalities in carbohydrate metabolism, have investigated 4-OH-Ile only as an effector of insulin-mediated or insulin-dependent pathways. The available prior art does not teach or suggest, however, fenugreek and/or 4-OH-Ile compositions that work synergistically or independently from insulin or insulin-mediated pathways. More particularly, there are presently no known prior art teachings or suggestions that 4-OH-Ile may affect the body by an insulin-independent mechanism. Stimulation of non-insulin mediated pathways may be desirable for targeting the utilization of carbohydrates in certain organ systems, (e.g., muscles, liver, etc.). Likewise, it may be desirable to avoid the general and/or systemic effects that may occur by stimulating the pancreas to produce and secrete insulin.

In addition, clinical studies conducted on fenugreek have focused on investigating a specific subfraction of the fenugreek seed (e.g., testa and endosperm) or, in the alternative, have focused on the specific effect of 4-OH-Ile in animals and humans with diabetes or a cholesterol disorder. Prior art directed to investigations of fenugreek seed subfractions have also failed to disclose or teach specific useful compositions.

Moreover, little or no attention has been given to the value of other bio-active compounds (e.g., free amino acids) present in fenugreek seeds, especially in augmenting the hypoglycemic and hypercholesterolemic actions of 4-OH-Ile. At least one prior art reference teaches there is little or no additional utility to be gained by extracting anything other than 4-hydroxyisoleucine from fenugreek seeds. Therefore, and as readily appreciated by those skilled in the art, a safer and more commercially practicable method for extracting compositions containing 4-OH-Ile and other bio-active components from fenugreek is needed. In addition, and as readily appreciated by those skilled in the art, novel compositions containing 4-OH-Ile for affecting the body by an insulin-independent mechanism are also needed. There is especially a need for a bio-active composition which has the blood glucose regulating activity of fenugreek but without the fiber or fat components of fenugreek seeds, prepared by methods which do not involve unacceptably toxic solvents or additives. There is a further need for a composition which comprises other fenugreek constituents, not limited to 4-OH-Ile, and comprising one or more of the following compounds: amino acids, alkaloids, glycosides, volatile oils, saponins, sapogenins, mannans, flavonoids, fatty acids, vitamins and provitamins, minerals, and carbohydrates.

Additionally, there is a need for methods of using such a fenugreek-based composition to improve glycemic control in humans and, more particularly, in individuals affected with disorders of carbohydrate metabolism tending to produce elevated blood sugar levels. There is a more specific need for novel compositions containing 4-OH-Ile for improving glucose tolerance and for regulating blood glucose by an insulin-independent mechanism.

Further, regardless of the underlying mechanism of the carbohydrate metabolism disorders referred to hereinabove, the most serious health consequences of these disorders (which include, for example, kidney failure, glaucoma and blindness, poor circulation in the extremities possibly leading to amputation, strokes and cardiovascular disease) result from the inability to establish and maintain blood sugar levels within a healthy range. Thus, there is considerable need for compositions and methods for improving glycemic control and glucose tolerance in individuals with carbohydrate metabolism disorders tending to cause excess blood sugar. Moreover, there is a particular need for such compositions and methods which can be used without resorting to exogeneous insulin. The novel compositions and methods of the present invention fulfill such long felt needs.

### SUMMARY OF THE INVENTION

The present invention comprises compositions and methods for improving glycemic control in humans and animals based upon naturally occurring compounds that may be derived, isolated, or extracted from seeds of the fenugreek plant (Trigonella foenum graecum). An embodiment of one such method is directed to improving glycemic control in animals or humans having a disorder of carbohydrate metabolism, particularly those disorders such as diabetes, prediabetes, and Syndrome X which are characterized by a tendency towards excess blood glucose levels or an inability to maintain blood glucose levels within an acceptable, desirable or healthy range as known in the art. A method contemplated by the present invention is directed to improving glucose tolerance in individuals afflicted with an insulin resistant disorder in carbohydrate metabolism. The method comprises the step of administering an effective amount of an amino acid complex comprising 4-hydroxyisoleucine (4-OH-Ile) and glutamate (inclusive of any chemical salts, anhydrides, or isomers of either of the foregoing).

In a further embodiment of the present invention, the amino acid complex is derived from fenugreek seeds, and a still further embodiment the complex includes amino acids which co-isolate with 4-OH-Ile and glutamate. The invention also encompasses processes of deriving the blood-sugar-regulating compositions by extraction methodologies from fenugreek seeds, and processes for defining and standardizing the content of key constituents of the fenugreek-seed-derived compositions.

Another embodiment of the present invention contemplates a method of managing blood sugar levels to avoid excess blood sugar, comprising a step of administering a composition comprising 4-OH-Ile and glutamate (inclusive of any chemical salts, anhydrides, or isomers of either of the foregoing). In an additional embodiment, the composition is derived, isolated, or extracted from fenugreek seeds (Trigonella foenum graecum).

In another embodiment, a composition contemplated by the present invention comprises 4-OH-Ile in an amount between about 20% and 30% by weight. In a further embodiment, the amount of a composition administered is selected to provide the 4-OH-Ile at a level of from 1 mg/kg body weight to 9 mg/kg body weight of the consuming individual. In yet a further embodiment, the amount of a composition administered provides the 4-OH-Ile at a level of from 2 mg/kg to 4 mg/kg body weight. In still another embodiment, a composition of the present invention comprises amino acids which co-isolate with 4-OH-Ile and glutamate. And in a still further embodiment of a composition of the present invention, the amino acids which co-isolate with 4-OH-Ile may include one or more of the following: arginine, aspartate, threonine, serine, glutamate, proline, glycine, alanine, cysteine, valine, methionine, isoleucine, leucine, tryptophan, phenylalanine, lysine, histidine, and tyrosine (inclusive of any chemical salts, anhydrides, or isomers of any of the foregoing).

Another embodiment of a method of the present invention for improving the ability of a person to achieve glycemic control, that is, his or her ability to maintain blood sugar levels within a desirable, healthy, or acceptable range as known in the art, comprises the step of administering an effective amount of an amino acid complex comprising 4-OH-Ile and glutamate (inclusive of any chemical salts, anhydrides, or isomers of either of the foregoing). The dosage amount may preferably be administered in oral form shortly before or together with a meal or snack to modulate any post-prandial increase in blood glucose. In one embodiment, an effective amount of an amino acid complex contemplated by the present invention is selected to provide 4-OH-Ile in an amount of from 1 mg/kg body weight to 9 mg/kg body weight. In a further embodiment, the amount of an amino acid complex of the present invention may be selected to provide 4-OH-Ile in an amount of from about 2 mg/kg body weight to about 4 mg/kg body weight. In yet another embodiment, the composition administered further comprises other fenugreek-derived components effective to regulate blood glucose by non-insulin-dependent pathways.

In one embodiment of a method for improving glycemic control in humans as contemplated by the present invention, a composition is administered is an amino acid complex comprising 4-OH-Ile, glutamate, together with one or more amino acids selected from the group consisting of alanine, arginine, aspartate, cysteine, gamma-aminobutyrate, glycine, histidine, isoleucine, leucine, lysine, methionine, ornithine, phenylalanine, praline, serine, threonine, tryptophan, tyrosine, and valine (inclusive of any chemical salts, anhydrides, or isomers of any of the foregoing).

Still another embodiment of a method comprises the step of administering a composition that may be derived, isolated or extracted from fenugreek seeds, comprising 4-OH-Ile, glutamate (inclusive of any chemical salts, anhydrides, or isomers of either of the foregoing), together with a component selected from the group consisting of: alkaloids, volatile oils, glycosides, saponins, sapogenins, flavonoids, mannans, vitamins and provitamins, minerals, botanicals, herbals, nucleotides, nutraceuticals, nutrients, pharmaceuticals, proteins, and the like. More specifically, the additional component may be selected from a group of compounds known to be present in fenugreek seeds, the group consisting of: acetylcholine, 25-alpha-spirosta-3,5-diene, 3,4,7-trimethylcoumarin, 3-hydroxy-4,5-dimethyl-2-furanone, 4-OH-Ile-lactone, 4-methyl-7-acetoxy-coumarin, 7-acetoxy-4-methylcoumarin, alpha-galactosidase, alpha-mannosidase, aluminum, arabinose, arachidic acid, behenic acid, beta-carotene, beta-mannanan, beta-sitosterol, biotin, carpaine, choline, coumarin, cyancobalamin, d-mannose, digalactosyl-myoinositol, dihydroactinidiolide, dihydrobenzofuran, dioscin, diosgenin, elemene, endo-betamannanase, fenugreekine, folacin, galactinol, galactomannan, gentianine, gitogenin, graecunin-h, graecunin-n, homoorientin, isovitexin, kaempferol, lecithin, lignin, luteolin, muurolene, myo-inositol, neotigogenin, niacin, nicotinic-acid, oleicacid, orientin, orientin arabinoside, p-coumaric-acid, palmitic acid, protopectin, pyridoxine, quercetin, raffinose, riboflavin, rutin, saponin, selenine, stachyose, stearic acid, thiamin, threonine, tigogenin, trigofoenosides, trigoforin, trigonelline, trigonellosides, trillin, verbascose, vicenin-1, vicenin-2, vitexin, vitexin-2′-o-p-coumarate, vitexin-7-glucoside, xanthophyll, yamogenin, yamogenin-3,26-biglycoside and yamogenin tetrosides.

Still another embodiment of the present invention contemplates a method of improving glucose tolerance in mammals having disordered carbohydrate metabolism leading to excess blood sugar levels, the method comprising the steps of administering an effective amount of a composition comprising an amino acid content including 4-OH-Ile and glutamate, wherein said 4-OH-Ile comprises an amount between about 60% and about 70% of a total weight of the amino acid content and the glutamate comprises an amount between about 6% and about 8% of the total weight of the amino acid content. In a further embodiment, the administered composition comprises between about 20% and about 30% of the 4-OH-Ile. In another embodiment, an administered composition of the present invention may comprise between about 23% and about 26% of the 4-OH-Ile.

A further embodiment of the present invention contemplates a method for improving glycemic control in humans comprising the step of administering an amount of a composition comprising an amino acid content including 4-OH-Ile and glutamate, wherein the 4-OH-Ile comprises an amount between about 60% and about 70% of a total weight of the amino acid content and the glutamate comprises an amount between about 6% and about 8% of the total weight of the amino acid content. In a further embodiment, the administered composition comprises between about 20% and about 30% of the 4-OH-Ile. In another embodiment, an administered composition of the present invention may comprise between about 23% and about 26% of the 4-OH-Ile. Additionally, the amino acid content of the composition may include one or more of the following amino acids selected from the group consisting of arginine in an amount between about 2.4% and about 2.7% of the total weight of the amino acid content, aspartate in an amount between about 4% and about 5% of the total weight of the amino acid content, cysteine in an amount between about 1% and about 2% of the total weight of the amino acid content, threonine in an amount between about 0.90% and about 1% of the total weight of the amino acid content, serine in an amount between about 4% and about 12% of the total weight of the amino acid content, glycine in an amount between about 2% and about 3% of the total weight of the amino acid content, alanine in an amount between about 3% and about 4% of the weight of the amino acid content, valine in an amount between about 1% and about 1.5% of the total weight of the amino acid content, methionine in an amount between about 0.35% and about 0.60% of the total weight of the amino acid content, isoleucine in an amount greater than 0.5% of the total weight of the amino acid content, and histidine in an amount between about 0.35% and about 0.40% of the total weight of the amino acid content.

A still further embodiment of the present invention contemplates a method for improving glycemic control in humans comprising the step of administering an amount of a composition comprising an amino acid content including 4-OH-Ile and cysteine, wherein the 4-OH-Ile comprises an amount between about 60% and about 70% of a total weight of said amino acid content and the cysteine comprises between about 1% and about 2% of the total weight of the amino acid content of the composition. In a further embodiment, the administered composition comprises between about 20% and about 30% of the 4-OH-Ile. In another embodiment, an administered composition of the present invention may comprise between about 23% and about 26% of the 4-OH-Ile. Additionally, the amino acid content of the composition may include one or more of the following amino acids selected from the group consisting of arginine in an amount between about 2.4% and about 2.7% of the total weight of the amino acid content, aspartate in an amount between about 4% and about 5% of the total weight of the amino acid content, glutamate in amount between about 6% and about 8% of said total weight of said amino acid content, threonine in an amount between about 0.90% and about 1% of the total weight of the amino acid content, serine in an amount between about 4% and about 12% of the total weight of the amino acid content, glycine in an amount between about 2% and about 3% of the total weight of the amino acid content, alanine in an amount between about 3% and about 4% of the weight of the amino acid content, valine in an amount between about 1% and about 1.5% of the total weight of the amino acid content, methionine in an amount between about 0.35% and about 0.60% of the total weight of the amino acid content, isoleucine in an amount greater than 0.5% of the total weight of the amino acid content, and histidine in an amount between about 0.35% and about 0.40% of the total weight of the amino acid content.

Another embodiment of the present invention contemplates a method for improving glycemic control in humans comprising the step of administering an amount of a composition comprising an amino acid content including 4-OH-Ile, glutamate, and aspartate, wherein the 4-OH-Ile comprises an amount between about 60% and about 70% of a total weight of the amino acid content, the glutamate comprises an amount between about 6% and about 8% of the total weight of the amino acid content, and the aspartate comprises an amount between about 4% and about 5% of the total weight of the amino acid content. In a further embodiment, the administered composition comprises between about 20% and about 30% of the 4-OH-Ile. In another embodiment, an administered composition of the present invention may comprise between about 23% and about 26% of the 4-OH-Ile. Additionally, the amino acid content of the composition may include one or more of the following amino acids selected from the group consisting of arginine in an amount between about 2.4% and about 2.7% of the total weight of the amino acid content, cysteine in an amount between about 1% and about 2% of the total weight of the amino acid content, threonine in an amount between about 0.90% and about 1% of the total weight of the amino acid content, serine in an amount between about 4% and about 12% of the total weight of the amino acid content, glycine in an amount between about 2% and about 3% of the total weight of the amino acid content, alanine in an amount between about 3% and about 4% of the weight of the amino acid content, valine in an amount between about 1% and about 1.5% of the total weight of the amino acid content, methionine in an amount between about 0.35% and about 0.60% of the total weight of the amino acid content, isoleucine in an amount greater than 0.5% of the total weight of the amino acid content, and histidine in an amount between about 0.35% and about 0.40% of the total weight of the amino acid content.

The present invention further encompasses processes for preparing compositions of the type administered from seeds of fenugreek. In one embodiment, a process contemplated by the present invention comprises the steps of preparing fenugreek seeds by separating the seed endosperm from the seed testa, performing a preliminary extraction on the prepared fenugreek endosperm and testa, and performing a secondary extraction on the fenugreek seeds. In one embodiment, the step of separating the endosperm from the testa comprises crushing and soaking the fenugreek seeds. In one embodiment, the step of performing the preliminary extraction comprises the steps of: (1) subjecting the prepared Fenugreek seeds to a solvent I to obtain a first seed residue and seed extract; (2) subjecting the collected seed residue to a solvent II to obtain a second seed residue and a concentrated extract; (3) further concentrating under vacuum; (4) cooling and settling to obtain a sediment of crude proteins and a supernatant; and (5) diluting the supernatant with de-ionized water;

In one embodiment of the present invention, the step of secondary extraction may comprise the steps of: (1) subjecting the preliminary supernatant to resin filtration with a macropore, non-polar or weakly polar cation ion exchange resin; (2) washing with de-ionized water; (3) progressive ethanol treatment using between about 10% and about 90% ethanol; (4) effluent collection; (5) pH adjustment to 1-6.5 with 6 Normal (N) hydrochloric acid (HCl); (6) treatment with 0.1-1 N ammonia solution; (7) effluent collection; (8) concentration under vacuum; (9) diluting with de-ionized water; (10) de-ammonification; and (11) drying to yield a composition of bio-active compounds comprising between about 20% and about 40% total protein and between about 10% and about 70% 4-OH-Ile by weight. In a further embodiment of the fenugreek extraction process, the resulting composition may have a 4-OH-Ile content of between about 20% and about 30%. Additionally, the resulting composition may comprise 4-OH-Ile between about 23% and about 26%.

Still another aspect of the invention is a quality control process for standardizing and quantifying the content of the bio-active compounds in a compositional product extracted from fenugreek seeds. One embodiment of a quality control process contemplated by the present invention may comprise the steps of: (1) providing a high-pressure liquid chromatography (HPLC) apparatus; (1) providing the following reagents: methanol, acetonitrile, sodium acetate trihydrate, glacial acetic acid, tetrafuran, OPA reagent, deionized water, and a 4-hydroxyisoleucine reference standard; (2) preparing analytes for examination in the HPLC apparatus including a mobile phase step, a standard preparation step, and a sample preparation step; (3) subjecting the analytes thus prepared to an HPLC injection program; and (4) observing and recording the peak spectra resulting from the injection program.

In one embodiment of the methods contemplated by the present invention, the compositions administered may be delivered and/or administered using any nutraceutical delivery form such as, for example and not by way of limitation, a tablet, capsule, powder, granule, microgranule, pellet, soft-gel capsule, controlled-release form, liquid, solution, elixir, syrup, suspension, emulsion, magma, gel, cream, ointment, lotion, transdermal, sublingual, ophthalmic form, nasal form, otic form, aerosol, inhalation form, spray, parenteral form (e.g., intravenous, intramuscular, subcutaneous), suppository, and the like.

In addition, the compositions of the present invention may be delivered and/or administered using any functional food delivery form, for example and not by way of limitation, a beverage, cereal, bar, bread, cracker, egg, juice, juice drink, milk, soft cheese, mineral water, pasta, pasta sauce, probiotic drink, soya product, spread, stimulation/energy beverage, yoghurt, baby and/or children's food, women's product, men's product, meal replacement, and the like.

### BRIEF DESCRIPTION OF THE DRAWINGS

The foregoing and other features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a plot depicting the comparison of a placebo glucose tolerance test performed on a normal human subject “S” challenged with a glucose solution placebo to a glucose tolerance test performed on subject “S” challenged with a glucose solution containing a 4-OH-Ile containing a composition administered in accordance with the methods contemplated by the present invention (hereinafter PROMILIN™) at a dose comprising 1 mg PROMILIN™/kg body weight;

FIG. 2 is a plot comparing the results of the placebo glucose tolerance test on human subject “S” to a first test in which subject “S” was challenged with a glucose solution containing a composition administered in accordance with the methods contemplated by the present invention (hereinafter PROMILIN™) at a dose comprising 4 mg PROMILIN™/kg body weight;

FIG. 3 is a plot comparing the results of the placebo glucose tolerance test on human subject “S” to a second glucose tolerance test of a composition administered in accordance with the methods contemplated by the present invention (hereinafter PROMILIN™) at a dose comprising 4 mg PROMILIN™/kg body weight;

FIG. 4 is a plot comparing the results of the placebo glucose tolerance test on human subject “S” to a third glucose tolerance test of a composition administered in accordance with the methods contemplated by the present invention (hereinafter PROMILIN™) at a dose comprising 4 mg PROMILIN™/kg body weight;

FIG. 5 is a plot comparing the results of the placebo glucose tolerance test on human subject “S” to a glucose tolerance test in which subject “S” was challenged with a glucose solution containing a composition administered in accordance with the methods contemplated by the present invention (hereinafter PROMILIN™) at a dose comprising 9 mg PROMILIN™/kg body weight;

FIG. 6 is a plot depicting the comparison of a placebo glucose tolerance test performed on a second normal human subject “C” challenged with a glucose solution placebo to a glucose tolerance test performed on subject “C” challenged with a glucose solution containing a composition administered in accordance with the methods contemplated by the present invention (hereinafter PROMILIN™) at a dose comprising 1 mg PROMILIN™/kg body weight;

FIG. 7 is a plot comparing the results of the placebo glucose tolerance test on human subject “C” to a first test in which subject “C” was challenged with a glucose solution containing a composition administered in accordance with the methods contemplated by the present invention (hereinafter PROMILIN™) at a dose comprising 4 mg PROMILIN™/kg body weight;

FIG. 8 is a plot comparing the results of the placebo glucose tolerance test on human subject “C” to a second glucose tolerance test of a composition administered in accordance with the methods contemplated by the present invention (hereinafter PROMILIN™) at a dose comprising 4 mg PROMILIN™/kg body weight;

FIG. 9 is a plot comparing the results of the placebo glucose tolerance test on human subject “C” to a third glucose tolerance test of containing a composition administered in accordance with the methods contemplated by the present invention (hereinafter PROMILIN™) at a dose comprising 4 mg PROMILIN™/kg body weight;

FIG. 10 is a plot comparing the results of glucose tolerance tests performed on a third normal human subject “D” challenged with a glucose solution placebo to those of a glucose tolerance test in which subject “D” was challenged with a glucose solution containing a composition administered in accordance with the methods contemplated by the present invention (hereinafter PROMILIN™) at a dose comprising 9 mg PROMILIN™/kg body weight;

FIG. 11 is a plot depicting the results of glucose tolerance tests in six human subjects each subjected to two different protocols as described in Example II, one in which a placebo was administered at the same time as the glucose challenge, and the other in which a composition administered in accordance with the methods contemplated by the present invention (hereinafter PROMILIN™) at a dose comprising 2 mg PROMILIN™/kg body weight;

FIG. 12 is a plot depicting post-challenge serum glucose levels in a second trial of human subjects given both control and glucose tolerance test protocols as described in Example II;

FIG. 13 is a plot depicting post-challenge insulin concentrations in the second trial;

FIG. 14 is a bar graph comparing the values of the area under the curve of post-challenge blood insulin levels for the placebo tests to those of the composition tests of FIGS. 11 and 12;

FIG. 15 is a bar graph illustrating the percentage (%) insulin increase by time period in glucose tolerance tests following the administration of the glucose challenge plus either placebo or a composition administered in accordance with the methods contemplated by the present invention (hereinafter PROMILIN™);

FIG. 16 is a graph depicting blood glucose levels as a function of time following a glucose challenge administered orally to fasted male Zucker diabetic rats treated with a placebo or with one of three dose levels of a composition administered in accordance with the methods contemplated by the present invention (hereinafter PROMILIN™);

FIG. 17 is a graph depicting plasma insulin levels as a function of time following a glucose challenge in fasted Zucker diabetic rats treated with a placebo or with one of three dose levels of a composition administered in accordance with the methods contemplated by the present invention (hereinafter PROMILIN™);

FIG. 18 is a graph depicting blood glucose levels as a function of time following a glucose challenge administered orally to fasted female Zucker diabetic rats treated with a placebo or with a composition administered in accordance with the methods contemplated by the present invention (hereinafter PROMILIN™);

FIG. 19 is a graph depicting plasma levels of 4-hydroxyisoleucine as a function of time following the administration of a placebo or a fenugreek-derived composition of the invention in male Zucker diabetic rats;

FIG. 20 is a bar graph depicting the distribution of fasting blood glucose levels in 4 groups of human subjects having Syndrome X in baseline trials;

FIG. 21 is a bar graph depicting the adjusted peak glucose level in four groups of human subjects having Syndrome X during glucose tolerance tests in a baseline trial and in a PROMILIN™ test trial;

FIG. 22 is a bar graph depicting the area under the blood glucose curve in glucose tolerance tests of the same four groups as in FIG. 21, for baseline and PROMILIN™ test trials;

FIG. 23 is a process flow diagram illustrating an embodiment of a method of the present invention for deriving, isolating, and/or extracting bio-active components from fenugreek seeds;

FIG. 24 is a process flow diagram illustrating an embodiment of a method of the present invention for preparing the fenugreek seeds as referenced in FIG. 23;

FIG. 25 is a process flow diagram illustrating an embodiment of a method of the present invention directed to the step of performing the preliminary extraction;

FIG. 26 is a process flow diagram illustrating an embodiment of a method of the present invention directed to the step of performing the secondary extraction as referenced in FIG. 23; and

FIG. 27 is a process flow diagram illustrating an alternative embodiment of a method of the present invention directed to the step of the secondary extraction as referenced in FIG. 23.

### DETAILED DESCRIPTION

It will be readily understood that the components of the present invention, as generally described and illustrated in the Figures herein, could be arranged and designed in a wide variety of difference compositional configurations and/or methods. Those of ordinary skill in the art will, of course, appreciate that various modifications to the details herein may be made without departing from the essential characteristics of the invention, as described. Thus, the following more detailed description of the embodiments of the compositions and methods of the present invention, as represented in the Figures and Tables, is not intended to limit the scope of the invention, as claimed, but it is merely representative of embodiments of the invention. The embodiments of the invention will be best understood by reference to the drawings and tables.

As outlined in the summary, the present invention is directed towards methods of using a composition that may be derived, isolated, or extracted from fenugreek seeds to regulate or maintain blood sugar levels in humans or animals having a disorder in carbohydrate metabolism, whereby falling into the realms of phytochemistry and pharmacognosy.

As appreciated by those skilled in the art, “pharmacognosy” may be defined as the investigation and evaluation of natural products in the search for new drugs and bio-active compositions. An important division of pharmacognosy is “phytochemistry,” which studies the chemistry of plants, plant processes and plant products. Folklore and knowledge of traditional remedies often provide the motivation for undertaking a phytochemical analysis of a particular plant or plant product, as was the case in this invention. As previously described, fenugreek may be said to fall into this condition, Nevertheless, it should be appreciated that a synthetically produced composition may also fall within the scope of the invention, if its composition is sufficiently similar to that of the composition in terms of key bio-active compounds as taught herein.

Fenugreek is recognized and has been known to have effects on lowering blood sugar and blood lipid levels. However, it was not until about thirty (30) years ago that systematic scientific investigations of fenugreek were initiated and subsequently, 4-hydroxyisoleucine was identified as a component of fenugreek. 4-OH-Ile is usually classified as an amino acid compound and has the following general formula:

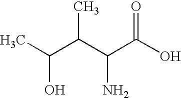

So that the reader may more clearly comprehend the true scope of the invention as illustrated by the following Examples and their associated Figures, the following section defines key terms used in the descriptions of these present embodiments.

Central to the methods of the present invention is a composition comprising 4-OH-Ile and one or more amino acids which may be used in a method for regulating blood glucose. The 4-OH-Ile containing composition administered in accordance with the methods contemplated by the present invention (hereinafter PROMILIN™) may be derived, isolated, or extracted from fenugreek (Trigonella foenum graecum). Its use in the following Examples should not be considered as limiting the scope or details of the administered composition of the present invention for improving glycemic control in any way beyond what is claimed, and having a composition comparable to any of the lots whose analysis is set forth in Examples V-VIII herein. It is believed that PROMILIN™ may further comprise one or more of the following compounds known to be present in fenugreek seeds: acetylcholine, 25-alpha-spirosta-3,5-diene, 3,4,7-trimethylcoumarin, 3-hydroxy-4,5-dimethyl-2-furanone, 4-OH-Ile-lactone, 4-methyl-7-acetoxy-coumarin, 7-acetoxy-4-methylcoumarin, alpha-galactosidase, alpha-mannosidase, aluminum, arabinose, arachidic acid, behenic acid, beta-carotene, beta-mannanan, beta-sitosterol, biotin, carpaine, choline, coumarin, cyancobalamin, d-mannose, digalactosyl-myoinositol, dihydroactinidiolide, dihydrobenzofuran, dioscin, diosgenin, elemene, endo-betamannanase, fenugreekine, folacin, galactinol, galactomannan, gentianine, gitogenin, graecunin-h, graecunin-n, homoorientin, isovitexin, kaempferol, lecithin, lignin, luteolin, muurolene, myo-inositol, neotigogenin, niacin, nicotinic-acid, oleicacid, orientin, orientin arabinoside, p-coumaric-acid, palmitic acid, protopectin, pyridoxine, quercetin, raffinose, riboflavin, rutin, saponin, selenine, stachyose, stearic acid, thiamin, threonine, tigogenin, trigofoenosides, trigoforin, trigonelline, trigonellosides, trillin, verbascose, vicenin-1, vicenin-2, vitexin, vitexin-2′-o-p-coumarate, vitexin-7-glucoside, xanthophyll, yamogenin, yamogenin-3,26-biglycoside and yamogenin tetrosides.

Further, the use of the embodiment designated PROMILIN™ in no way excludes the potential to synthetically construct a composition comprising the bio-active compounds found in PROMILIN™ in comparable amounts, and using such a synthetic composition to achieve the methods of glycemic control as described herein. Such would be considered to fall within the scope of the instant invention.

In summary, use of the term PROMILIN™ is not meant to limit the scope of the composition administered in the claimed methods for improving glycemic control, nor to limit the scope of the methods of improving glycemic control and improving glucose tolerance in any manner other than that set forth in the attached claims.

The term “disorder of carbohydrate metabolism” refers to conditions in which the affected individual is unable to generally maintain his or her blood sugar levels within a desirable healthy range, or whose blood sugar levels tend to fluctuate outside the healthy range to a significant degree, or whose glucose tolerance as assessed by means known in the art is impaired, all as judged by physicians and others skilled in the art. Such disorders of carbohydrate metabolism are deemed to include (but are not limited to) diabetes mellitus; insulin-dependent diabetes (also referred to as IDD or type I diabetes); non-insulin-dependent diabetes (also referred to as NIDD or type II diabetes); pre-diabetes; insulin resistance; and Syndrome X (which is defined in part by insulin resistance, a tendency to excess abdominal fat, and elevated fasting blood sugar levels).

Disorders of carbohydrate metabolism are classified as either insulin dependent (alternatively referred to as insulin responsive or insulin sensitive), or as insulin resistant (alternatively referred to as insulin independent), as generally known in the art. Insulin is a hormone that plays a critical role in normal carbohydrate metabolism in humans and in mammals generally. Insulin dependent conditions can be ameliorated by either stimulating additional endogeneous insulin secretion, or by providing additional functional insulin in an exogeneous manner, as known in the art. For purposes of this application of this application, the term “insulin resistant disorder of carbohydrate metabolism” is defined as including all those conditions in which merely increasing the blood level of insulin does not provide satisfactory control of blood sugar levels; such conditions include pre-diabetes, Syndrome X, and late onset Type II diabetes.

For purposes of this application, the term “glycemic control” means the ability to maintain blood sugar levels within an acceptable, desirable, or healthy range as known in the art, in the course of normal life. As well known by those who treat diabetes, many factors influence a person's blood sugar level in the course of normal living. These factors include fasting, eating, drinking alcohol, and exercise; all of which can cause blood sugar levels to fluctuate in both normal individuals and in those having a disordered carbohydrate metabolism.

As known in the art, a common method for assessing the degree of glycemic control (the functioning of carbohydrate metabolism) in humans and animals is the “glucose tolerance test.” For purposes of this application, the term “glucose tolerance test” will be used to refer to any test in which a subject undergoes a period fasting followed by administration of a sugar (i.e., normally glucose) challenge, and wherein the subject's blood glucose levels are measured just prior to the glucose challenge and at intervals thereafter.

The term “glucose tolerance” refers to the degree to which a subject is able to keep his or her blood glucose levels within a normal or healthy range in the period following the challenge. Thus, improved glucose tolerance means an enhanced ability to maintain blood glucose levels within a desired, acceptable or healthy range in the face of the fluctuations occurring in response to activities of daily living. Conversely, the terms glucose intolerance or impaired glucose tolerance signify a reduced ability to maintain blood sugar levels within acceptable levels.

Additionally, a number of terms relating to the chemistry of the central composition are defined as follows for purposes of the application.

“Amino acids” may be defined as organic acids containing both an amino and carboxylic acid functional group, and which a portion of the non-acid hydrogen has been replaced by one or more amino groups. An amino acid may therefore have both basic and acidic properties. More than three hundred amino acids are known to occur in nature, however, only twenty amino acids are used in the synthesis of protein chains.

These twenty amino acids have the absolute configuration of L-glyceraldehyde and are therefore labeled as L-α amino acids. L-α amino acids include alanine, arginine, asparagine, aspartic acid (also referred to as aspartate), cysteine, glutamic acid (also referred to as glutamate), glutamine, glycine, histidine, isoleucine, leucine, lysine, methionine, phenylalanine, proline, serine, threonine, tryptophan, tyrosine, and valine.

Moreover, nine of the twenty amino acids cannot be manufactured in vivo by animals and must be supplied through the hydrolysis of dietary protein. These nine amino acids may be defined as essential amino acids and include arginine, isoleucine, leucine, lysine, methionine, phenylalanine, threonine, tryptophan, and valine.

Certain L-α amino acids have chemical and physical properties based on their respective side chains. A non-acidic hydrogen may also be replaced by a side chain with a chemical functional group. A “functional group” may be defined as an atom or group of atoms, acting as a unit, that has replaced a hydrogen atom in a hydrocarbon molecule and whose presence imparts characteristic chemical and physical properties to the hydrocarbon molecule. Characteristic chemical and physical properties may include, without limitation, acidity, basicity, aromaticity, hydrophilicity, and hydrophobicity.

Functional groups may include, without limitation, aliphatic groups, acid groups, hydroxyl groups, basic groups, aromatic groups, and sulfur groups. For example, the side chains of isoleucine, leucine, and valine all contain branched-chain aliphatic groups. These three amino acids are therefore commonly referred to as branched-chain amino acids (BCAAs). Other amino acids contain hydroxylic groups (e.g., serine, threonine, tyrosine), sulfur atoms (e.g., cysteine, methionine), acid groups or their amides (e.g., aspartic acid, asparagine, glutamic acid, glutamine), basic groups (e.g., arginine, lysine, histidine), aliphatic groups (e.g., alanine, glycine), and aromatic rings (histidine, phenylalanine, tyrosine, tryptophan). Proline is unique from other amino acids in that it may form an imino acid structure.

Amino acids serve many important roles in the homeostasis and physiological functions in both humans and animals. BCAA's are important to muscle growth and may account for the most common amino acids in muscle tissue. They are also important to the synthesis of neurotransmitters for the nervous system. Amino acids containing basic groups (i.e., arginine, lysine, histidine) are also important to muscle growth. These amino acids may also serve as a precursor to growth hormone and may have an important role in the transport, storage, and elimination of ammonia from the body. Glycine may be used to form porphyrins, which are used in the transport of oxygen. Glycine, aspartate, and glutamine may be used in the synthesis of purine and pyrimidine bases for use in nucleotides and management of genetic material. Arginine and glycine are important components in the synthesis of creatine, which is important for muscle function. As appreciated, tryptophan, tyrosine, and histidine may be used to form many important neurotransmitters (e.g., serotonin, melatonin, catecholamines, dopamine, and histamine).

A number of other amino acids that may have important homeostasis and physiological functions include homocysteine, homoserine, homocysteine, carnitine, ornithine, citrulline, arginosuccinic acid, 3,4-dihydroxyphenylalanine (DOPA), gamma-aminobutyric acid (GABA), glutathione, taurine, and thyroxine as well as many others. Ornithine and GABA are known to occur in fenugreek. Trimethylhistidine is a quaternary ammonium compound that has a structure similar to the amino acid histidine and may be found in fenugreek.

Several other bio-active compounds may also be isolated from fenugreek, including, for example: 25-alpha-spirosta-3,5-diene, 3,4,7-trimethylcoumarin, 3-hydroxy-4,5-dimethyl-2-furanone, 4-hydroxyisoleucine-lactone, 4-methyl-7-acetoxycoumarin, 7-acetoxy-4-methylcoumarin, acetyl-choline, alpha-galactosidase, alpha-mannosidase, aluminum, arabinose, arachidic-acid, behenic-acid, beta-carotene, beta-mannanan, beta-sitosterol, biotin, carpaine, choline, coumarin, cyancobalamin, d-mannose, digalactosylmyoinositol, dihydroactinidiolide, dihydrobenzofuran, dioscin, diosgenin, elemene, endo-beta-mannanase, fenugreekine, folacin, galactinol, galactomannan, gentianine, gitogenin, graecunin-h, graecunin-n, homoorientin, isovitexin, kaempferol, lecithin, lignin, luteolin, muurolene, myo-inositol, neotigogenin, niacin, nicotinic acid, oleic acid, orientin, orientin arabinoside, p-coumaric acid, palmitic acid, protopectin, pyridoxine, quercetin, raffinose, riboflavin, rutin, saponin, selenine, stachyose, stearic acid, thiamin, threonine, tigogenin, trigofoenosides, trigoforin, trigonelline, trigonellosides, trillin, verbascose, vicenin-1, vicenin-2, vitexin, vitexin-2′-o-p-coumarate, vitexin-7-glucoside, xanthophyll, yamogenin, yamogenin-3,26-biglycoside, and yamogenin tetrosides.

Many of these other bio-active compounds are alkaloids, glycosides, volatile oils, saponins, sapogenins, galactomannans, flavonoids, fatty acids, provitamins and vitamins, minerals, and carbohydrates. “Alkaloids” may be defined as organic bases that contain nitrogen and usually contain oxygen. They are found in some seed plants and may be in the form of salts with acids (e.g., as citric, oxalic, or sulfuric acid). Alkaloids may be colorless and well crystallized and bitter tasting. They tend to be complex in structure with at least one nitrogen atom in a ring (e.g., as a pyrrole, quinoline, or indole ring), and optically and biologically active.

“Glycosides” may be defined as any of a large class of natural or synthetic compounds that are acetal derivatives of sugars. When hydrolyzed, glycosides may yield one or more molecules of a sugar and often a noncarbohydrate. Glycosides may also exist as a mixed acetal, which contains a cyclic form of a glycose, a hemiacetal, and which may be classified as a furanoside or pyranoside according to the size of the ring of the glycose or as an alpha glycoside or a beta glycoside according to the optical rotation.

“Volatile oils” may be defined as any oil which readily vaporizes when exposed to air at ordinary temperatures (i.e., room temperature). Volatile oils are sometimes referred to as essential oils. They may be any of a large class of oils of vegetable origin that impart odor and often other characteristic properties to plants. Volatile oils may be obtained from various parts of the plants (e.g., seeds, flowers, leaves, bark) by steam distillation, expression, or extraction. Typically, volatile oils are mixtures of compounds (as terpenoids, aldehydes, or esters). Volatile oils are often used in the production of perfumes, flavoring materials, and pharmaceutical preparations. Fenugreek is known to contain the following volatile oils: 3-hydroxy-4,5-dimethyl-2-furanone, dihydrobenzo-furan, dihydroactinidiolide, elemene, murolene, and selinene.

“Saponins” may be defined as any of numerous glycosides that occur in many plants, saponins may be characterized by their properties of foaming in water solution and producing hemolysis when solutions are injected into the bloodstream. When hydrolyzed, saponins may yield a triterpenoid or steroid sapogenin and one or more sugars (e.g., glucose, galactose, xylose). As appreciated, fenugreek may include the following saponins: 25-alpha-spirosta-3,5-diene and dioscin.

“Sapogenins” may be defined as the non-sugar portion of a saponin obtained by hydrolysis. In a few cases, sapogenins may be found free in plants. Sapogenins may be characterized by either a triterpenoid, usually pentacyclic structure (e.g., quillaic acid) or by a steroid structure usually having a spiro acetal side chain (e.g., diosgenin). Steroidal sapogenins may be useful as starting materials in the synthesis of steroidal hormones. One sapogenin, diosgenin, with the empirical formula C₂₇H₄₂O₃, may be obtained in Mexico from locally available yams (e.g., Mexican Wild Yam) and may be used as a starting material for the synthesis of steroid hormones (e.g., cortisone, contraceptive hormones, anabolic hormones, dehydroepiandrosterone (DHEA)). Fenugreek is known to contain the following sapogenins: diosgenin, fenugreekine, gitogenin, neotigogenin, tigogenin and yamogenin

“Galactomannans” may be defined as any of several polysaccharides that occur especially in seeds (e.g., locust beans). When hydrolyzed, galactomannan may yield galactose and mannose. Galactomannans may be characterized as soluble fiber. “Soluble fiber” may be defined as coarse, mostly indigestible plant matter, consisting primarily of polysaccharides, that when eaten stimulates intestinal peristalsis. Fiber may also be referred to as roughage, coarse fodder or bulk.

“Flavonoids” may be defined as compounds which are related to flavone, a colorless crystalline ketone C₁₅H₁₀O₂ or any of the derivatives of this ketone many of which (e.g., chrysin) occur as yellow plant pigments often in the form of glycosides (e.g., apiin). As appreciated, fenugreek seed may contain the following flavonoids: homoorientin, orientin, quercetin, trigoforin, trillin, vicenin-1, vicenin-2, vitexin, isovitexin, and luteolin.

“Fatty acids” may be defined as any of the series of saturated aliphatic monocarboxylic acids with the general empiric formula of CₙH₂ₙ₊₁COOH (e.g., acetic acid, lauric acid, myristic acid, palmitic acid, stearic acid, arachidic acid, behenic acid, lignoceric acid), or unsaturated aliphatic monocarboxylic acids (e.g., palmitoleic acid, oleic acid, linoleic acid, arachidonic acid). Fatty acids occur naturally usually in the form of esters in fats, waxes, and oils. Fatty acids may also be in the form of glycerides in fats and fatty oils. Fatty acids, in almost all cases, contain an even number of carbon atoms most commonly between from about 12 to about 24 carbon atoms in the higher acids. Fatty oils may sometimes be referred to as fixed oils. These oils are generally in liquid form at ordinary temperatures.

“Vitamins” may be defined as organic compounds which are required in small quantities for normal metabolism. Vitamins cannot be synthesized by the body in adequate amounts and act typically in the regulation of various metabolic processes, but do not provide energy or serve as building units. Biochemical precursors to vitamins are often referred to as provitamins. Fenugreek may contain one or more of the following vitamins and pro-vitamins: acetylcholine, beta-carotene, choline, cyancobalamin, folacin, niacin, nicotinic acid, pyridoxine, riboflavin, thiamine, and xanthophyll.

“Minerals” may be defined as solid homogeneous crystalline chemical elements or compounds that result from inorganic processes of nature. Minerals have a characteristic crystal structure, color, and hardness. They may exist in a chemical composition or range of compositions. Minerals may be referred to as inorganic elements, and may be essential to the nutrition of humans, animals, and plants. The following minerals may be found in fenugreek: calcium, chromium, cobalt, copper, iron, magnesium, manganese, phosphorous, potassium, selenium, silicon, sodium, sulfur, tin, and zinc.

“Carbohydrates” may be defined as any of a group of organic compounds that includes sugars, starches, celluloses, and gums. Carbohydrates may serve as a major energy source in the diet in humans and animals. Carbohydrates may be produced by photosynthetic plants and contain only carbon \textcircled{C}, hydrogen (H), and oxygen (O), usually in the ratio 1:2:1, respectively.

The term “saccharide” may sometimes be used to describe a sugar. A saccharide may include any of a series of compounds consisting of carbon, hydrogen, and oxygen in which the atoms of the latter two elements, H and O, are in the ratio of 2:1, respectively, for example, C₆H₁₀O₅ and C₅H₁₀O₅Saccharides may also be classified according to how many units or components they contain. A monosaccharide may be characterized as the simplest form of saccharide and may include those carbohydrates which cannot be hydrolyzed into a simpler form. Monosaccharides may include organic compounds with between three and nine carbon atoms. Disaccharides may be defined as compounds which, upon hydrolysis, yield two monosaccharides that may be the same or different. Oligosaccharides may be defined as compounds which, upon hydrolysis, yield between three and six monosaccharide units that may be the same or different. A polysaccharide may be defined as compounds which, upon hydrolysis, yield more than six monosaccharide units that may be the same or different.

Examples I-III illustrate the effectiveness of embodiments of the methods of the present invention for improving glycemic control, including effective dosing of the 4-OH-Ile containing compositions (hereinafter PROMILIN™) taught and claimed herein.

### EXAMPLE I

### Non-Debitterized Fenugreek Seed Extract in Humans—1 mg 4-OH-Ile/kg of Body Weight

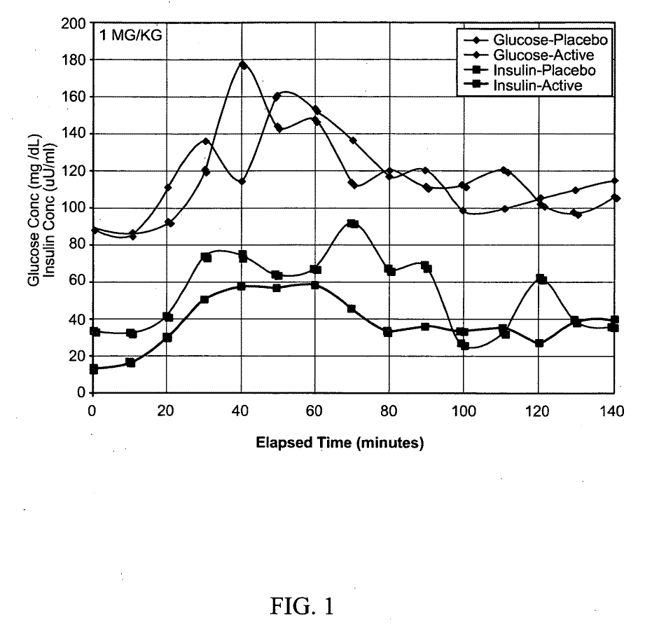

Generally referring now to FIGS. 1-10, oral glucose tolerance tests similar to those used to diagnose diabetes and gestational diabetes were conducted with three healthy male human subjects with no history of diabetes or carbohydrate metabolism dysfunction. Prior to each test, the subjects fasted overnight. At a time designated as time zero, an oral glucose challenge was administered as a solution containing 75 grams of glucose in 300 mL water. At intervals of 15, 30, 60, 120, and 180 minutes thereafter, blood samples were taken and blood glucose levels and insulin levels were determined by well-known methods for each sample (insulin content was measured using Radioimmunassay (RIA) at the School of Pharmacy at University of Montana). In addition a blood sample was drawn 30 minutes prior to administration of the glucose test solution. Two types of tests were performed with each of the three subjects.

In the first type of test, which will be termed the placebo test, the fasted subject was challenged with a plain glucose solution (e.g., 75 g) as in an ordinary OGTT. In the second type of test, the subject was administered a bio-active solution which consisted of the same glucose solution to which PROMILIN™ (a non-debitterized fenugreek extract containing 4-hydroxyisoleucine and other constituents as described herein) was added in an amount selected such that the 4-OH-Ile content amounted to one of the specific dose levels. These dose levels were, as follows: 1 mg PROMILIN™/kg of the subject's body weight; 4 mg PROMILIN™/kg; and 9 mg PROMILIN™/kg. The tests using the non-debitterized fenugreek extract containing 4-OH-Ile and other constituents will be referred to as PROMILIN™ tests

Blood glucose and insulin levels from these tests are graphically summarized in FIGS. 1-10, where the “placebo portion” data points represent those tests where the subjects received glucose solution alone, and the “active portion” data points represent those tests where the subjects received PROMILIN™ in addition to glucose. Before discussing the results obtained in these OGTT tests, it is emphasized that while the PROMILIN™ doses administered were standardized to particular levels of 4-OH-Ile, PROMILIN™ also comprises one or more amino acids (notably glutamate), and may also include other components as more fully described elsewhere in this application.

Referring to FIGS. 1-5, these charts depict the results of five placebo tests and five PROMILIN™ tests for the first human subject (Subject “S”). FIG. 1 is a plot of insulin levels and blood glucose levels (vertical axis) versus post-challenge elapsed time (horizontal axis), for one placebo test and one PROMILIN™ test at a PROMILIN™ dose providing 1 mg PROMILIN™/kg of body weight. In the PROMILIN™ test, insulin levels were significantly higher post-challenge than in the placebo test. However, as can be seen, there was at most a slight reduction in post-challenge glucose levels for the PROMILIN™ test as compared to the placebo.

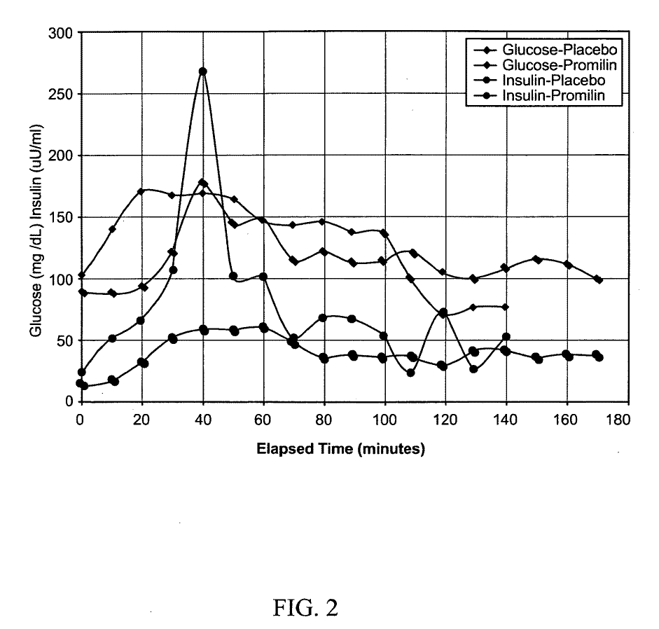

Turning to FIGS. 2, 3 and 4, these graphs depict the results of three pairs of placebo and PROMILIN™ tests in Subject “S” at the same PROMILIN™ dose, which provided 4 mg PROMILIN™/kg of body weight. Referring specifically to FIG. 2, there was a strong increase in insulin levels in the PROMILIN™ test as compared to the placebo, but again the effect of PROMILIN™ on glucose levels was less, although more pronounced than in the 1 mg PROMILIN™/kg of body weight test.

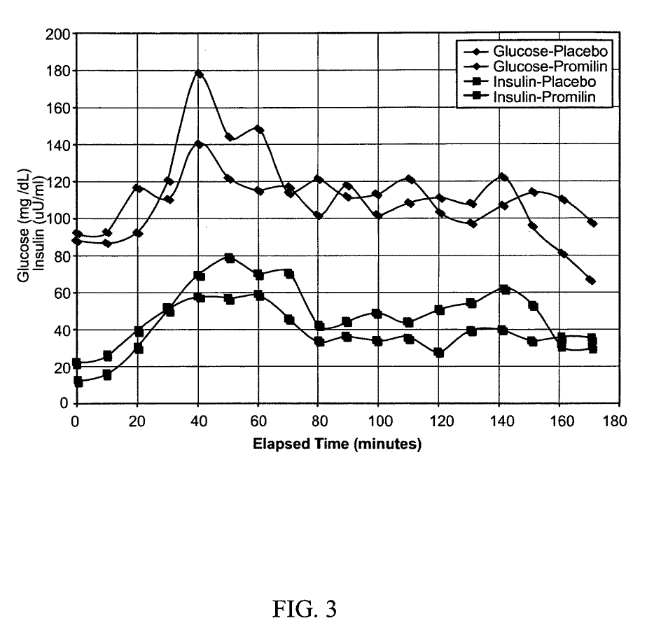

Moving now to FIG. 3, in this case the same 4 mg PROMILIN™/kg of body weight dose resulted in a distinct increase in insulin levels over placebo, but not as great as in the test illustrated in FIG. 2. However, despite the more modest insulin response, there was a very marked reduction in post-challenge glucose levels in the PROMILIN™test as compared to placebo.

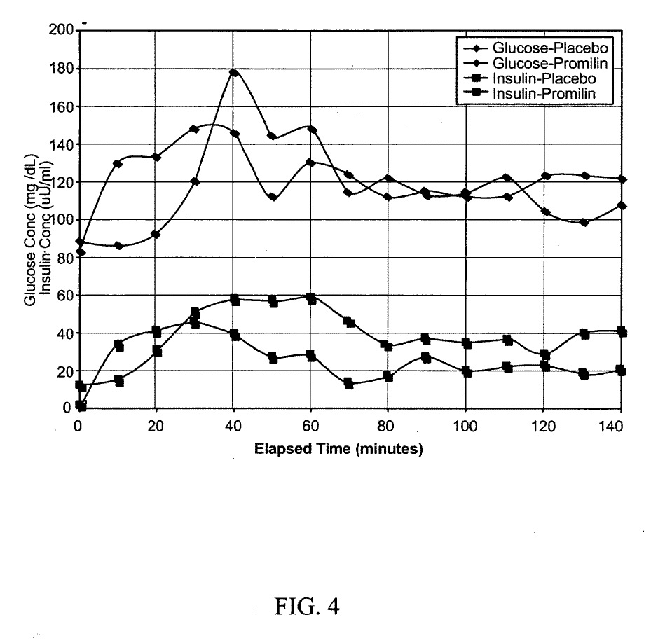

Finally, looking now at FIG. 4, there is again a significant increase in insulin in the PROMILIN™ test; however the effect on glucose level is unclear. If the post-challenge glucose results are considered in terms of area under the curve, which is a metric commonly used to assess the results of an OGTT, the effect of PROMILIN™ on glucose levels appears to have been minimal in the test depicted in FIG. 4

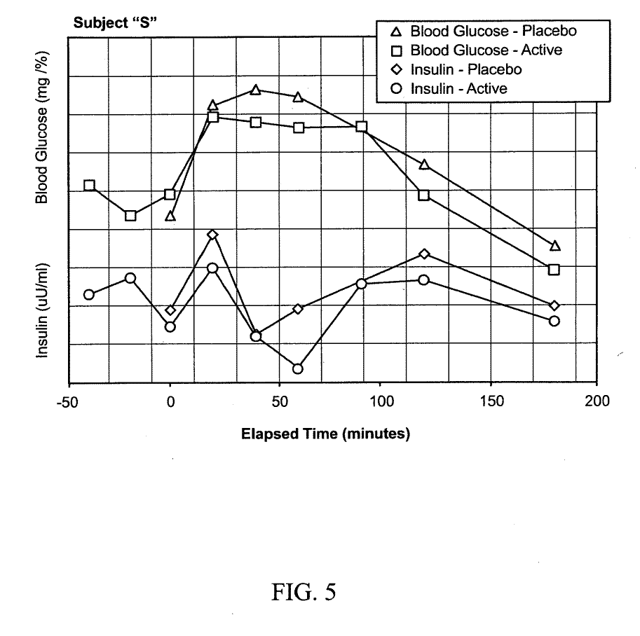

Turning now to FIG. 5, which depicts the results of a fifth placebo test compared to the last PROMILIN™ dose level studied in Subject “S”, which provided 9 mg PROMILIN™/kg of body weight, here the effects of PROMILIN™ on both insulin levels and glucose levels was rather small when compared to the PROMILIN™ response at the 4 mg PROMILIN™/kg of body weight dose. The reason for this is unknown, but a similar biphasic response pattern has been observed in other experiments. (See e.g., Example IV infra.)

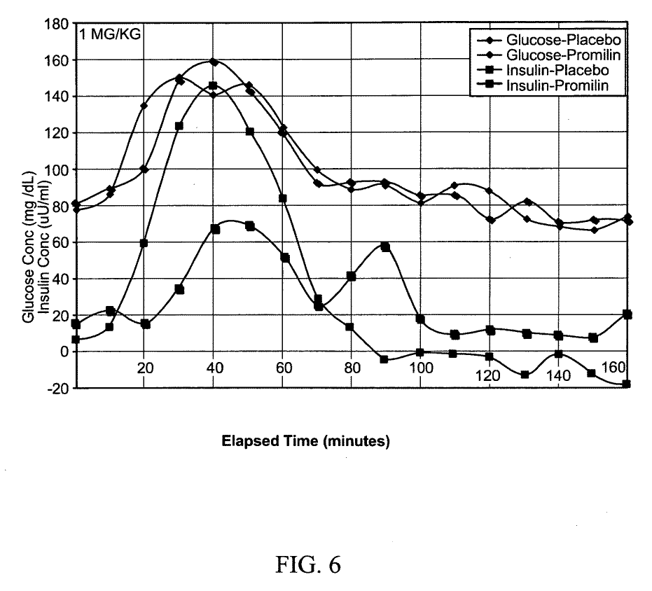

The results obtained with the second human subject, Subject “C”, are depicted in FIGS. 6 through 9. As shown, FIG. 6 depicts the results of OGTT tests on Subject “C” with and without a dose of PROMILIN™ standardized to deliver 1 mg PROMILIN™/kg of body weight. It can be seen that while there was a large increase in insulin levels in the PROMILIN™ test over that in the placebo, there was comparatively little difference in post-challenge glucose levels.

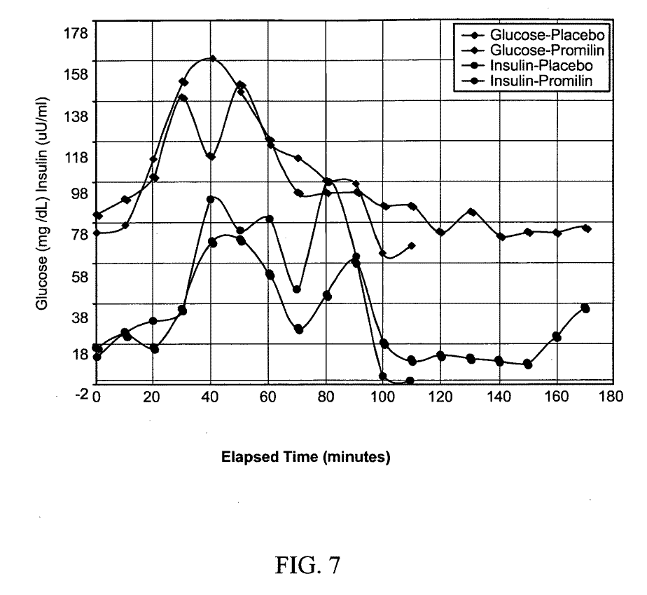

Referring now to FIGS. 7, 8 and 9, these depict the results of three PROMILIN™ tests in Subject “C” at the 4 mg PROMILIN™/kg of body weight/kg dose as compared to the placebo test. Referring to FIG. 7, there was a significant increase in insulin levels in the PROMILIN™ test as compared to the placebo, but the effect of PROMILIN™ on glucose levels is relatively small, although greater than in the 1 mg PROMILIN™/kg of body weight test.

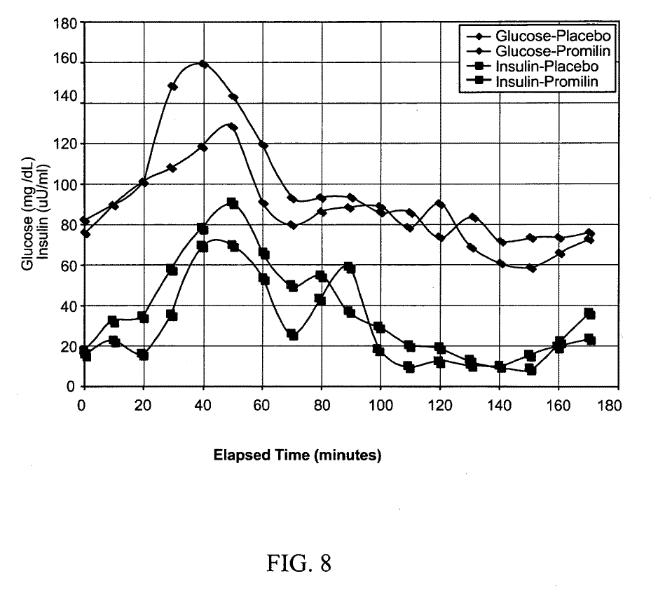

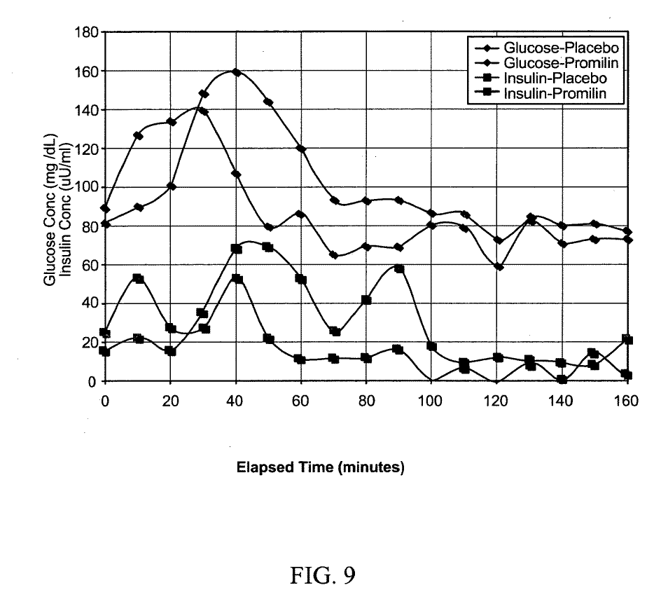

Turning now to FIG. 8, in this case the same 4 mg PROMILIN™/kg of body weight dose produced significant increase insulin levels over placebo, comparable to that in FIG. 7, along with a substantial reduction in post-challenge glucose levels Finally, looking at FIG. 9, here there was a very substantial increase in the PROMILIN™ test, together with a similarly substantial reduction in post-challenge glucose levels, as compared to the placebo.

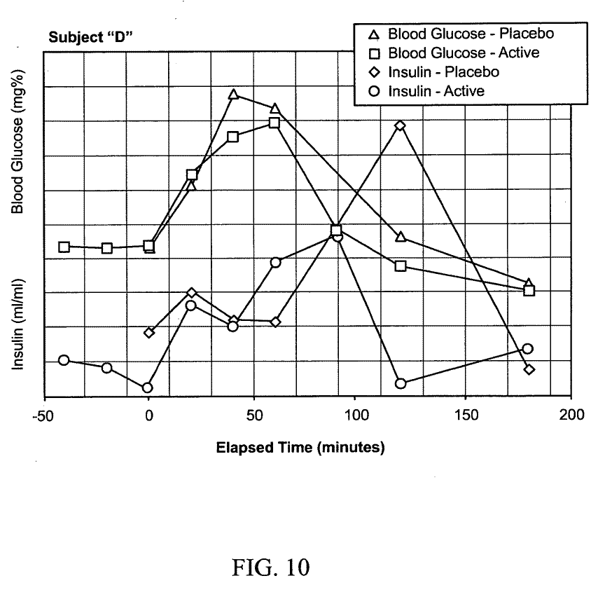

Lastly, we turn to FIG. 10 which depicts the results of a placebo test and a PROMILIN™ test in a third human subject (Subject “D”), at a dose of 9 mg PROMILIN™/kg of body weight. Although there was one very high data point for insulin level in the PROMILIN™ test, the remainder of the insulin response curve appears little different than that of the placebo. Similar to the 9 mg PROMILIN™/kg of body weight test in Subject S (see FIG. 5), the PROMILIN™-mediated reduction in glucose levels was rather small.

Considering the foregoing test results as a whole, it can be seen that the 4 mg PROMILIN™/kg of body weight dosage level had marked effects on glucose tolerance in the two subjects tested (Subjects “S” and “C”). Interestingly, the higher 9 mg PROMILIN™/kg of body weight dose had little effect on either the insulin response or the glucose response in both of the subjects tested (e.g., Subjects “S” and “D”).

### EXAMPLE II

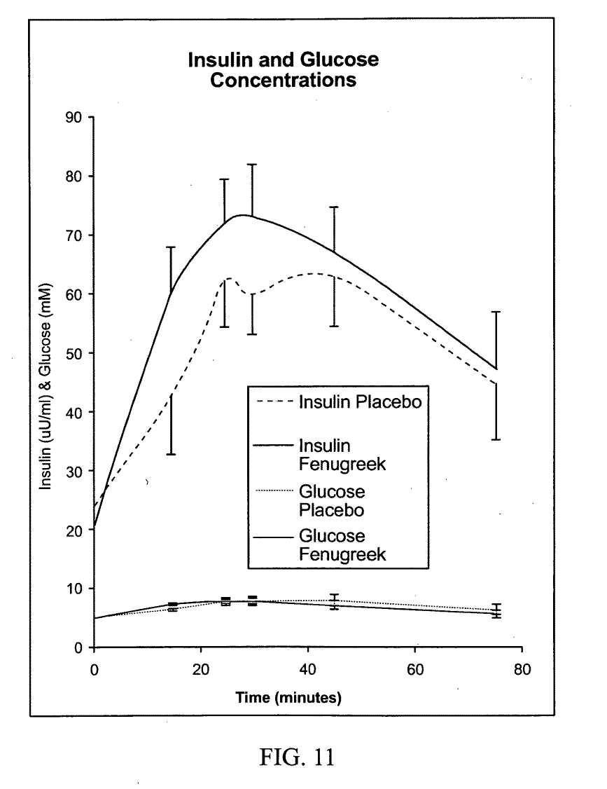

Generally referring now to FIGS. 11-15, tests were conducted with six healthy male subjects who were recreationally active, engaging in resistance or aerobic training on an average of three days a week. Subjects underwent a two day trial to evaluate the effect of PROMILIN™ on glucose metabolism. The study protocol was essentially a glucose tolerance test (GTT) of the type known in the art for use to diagnose diabetes, insulin resistance, and similar conditions. As known in the art, such studies typically involve (1) having the subject fast (abstain from food and alcohol) for from 12 to 24 hours (overnight is common), followed by (2) measuring the subject's blood glucose level at a time designated time zero, (3) administering a glucose challenge, and (4) determining blood glucose level at various intervals for several hours post-challenge. The instant study further included measuring blood insulin levels in the samples used to determine blood glucose (GTT+insulin). In addition, the instant study contained two branches: the control branch in which the GTT+insulin was conducted using a glucose challenge of 1.8 g glucose/kg of body weight administered together with a placebo, and a PROMILIN™ test branch in which, in addition to the glucose challenge, at time zero the subject was also given PROMILIN™ at a selected dose.

For purposes of this study the PROMILIN™ dose was defined on the basis of the content of 4-OH-Ile, but this was done for standardization purposes only and is meant neither to limit the scope of the compositions of the present invention (which comprises one or more other components and constituents as described herein), nor to imply that the content of 4-OH-Ile is solely responsible for the biological activity of PROMILIN™.

As appreciated, all subjects were administered the control test first in order to minimize carryover effect.

### Methodology:

Subjects arrived at the lab following an overnight fast (i.e., at least 12 hours). Experimental trials were separated by no less than 24 hours. An indwelling venous catheter (20 gauge) was inserted into an anticubital arm vein of each subject and kept patent using a continuous saline intravenous drip. Following the collection of a baseline blood sample, each subject received an oral glucose tolerance test beverage (i.e., 1.8 grams/kg BW), plus three tablets consisting of either a placebo (control) or PROMILIN™, in an amount selected to provide 2.0 mg PROMILIN™/kg of body weight.

Following the initial ingestion of the glucose tolerance test beverage and supplement, subsequent whole blood samples were collected from the subject at time zero (i.e., before challenge) and at times 15, 25, 30, 45, 60, and 75 minutes (i.e., minutes post-challenge). Whole blood samples were placed in plain tubes and centrifuged for about 20 minutes at approximately 3000 rpm. The resultant serum was separated and stored at −30° C. for subsequent analysis.

Analysis of blood glucose was completed in duplicate by means of a Milton Roy spectrophotometer using a commercially available assay kit (e.g., Sigma, glucose infinity reagent). Insulin was assayed using a commercially available enzymatic kit (e.g., DRG, International—Human Insulin EIA-2935). All samples were measured in duplicate. The average coefficient of variation for the glucose assay was 2.1±1.8%. The average coefficient of variation for the insulin assay was 8.9±4.0%.

### Results:

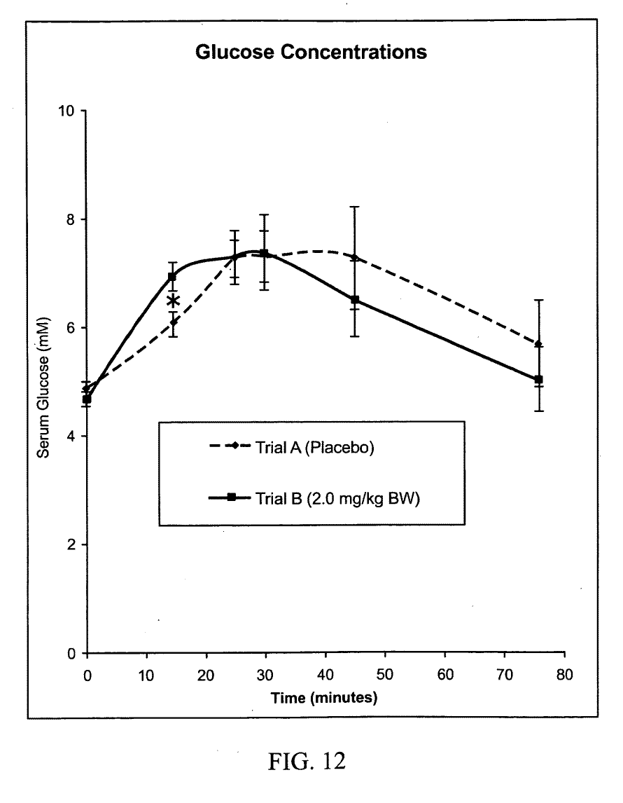

With reference to FIG. 11, a graph is shown of the insulin and glucose concentrations plotted over the time course of the post-challenge sample times. As illustrated, the vertical axis (i.e., ordinate) references insulin (μIU/mL) and glucose (mM) units and the horizontal axis (i.e., abscissa) references time (minutes). The confidence intervals for the plotted values are indicated by brackets. The top two curves illustrate the changes in glucose concentration following administration of an oral glucose bolus with placebo (dashed line) or with PROMILIN™ (solid line). The bottom two curves illustrate the changes in insulin concentration following administration of an oral glucose bolus with placebo (dashed line) or with PROMILIN™ (solid line). These data are further discussed below and in FIGS. 12-15.

Referring now to FIG. 12, a graph is shown of the serum glucose concentration plotted over the time course of the post-challenge sample times. As illustrated, the vertical axis (i.e., ordinate) references serum glucose concentrations measured in milliMolar (mM) units. The horizontal axis (i.e., abscissa) references the sample times from time zero (0) (i.e., just before receiving glucose and the experimental challenge) to time 75 minutes. The confidence intervals for the plotted values are indicated by brackets. The data for the serum glucose response following placebo challenges are indicated by a dashed line and the data for serum glucose response following challenges with a novel composition of bio-active compounds derived, isolated, and/or extracted from fenugreek seeds are indicated by a solid line. The 15 minute time point is marked with an asterisk (i.e., *) and indicates a significant difference in serum glucose concentrations between feedings with a novel composition of bio-active compounds derived, isolated, and/or extracted from fenugreek seeds and feedings with a placebo.

There were no significant differences between the trials for blood glucose at other time points. As shown, the total area under the curve (AUC) for blood glucose was similar for both trials during the initial 0 to 45 minute time segment. However, the total AUC was significantly lower during the experimental trial for the 45 to 75 minute time segment and for the overall 0 to 75 minute time segment.

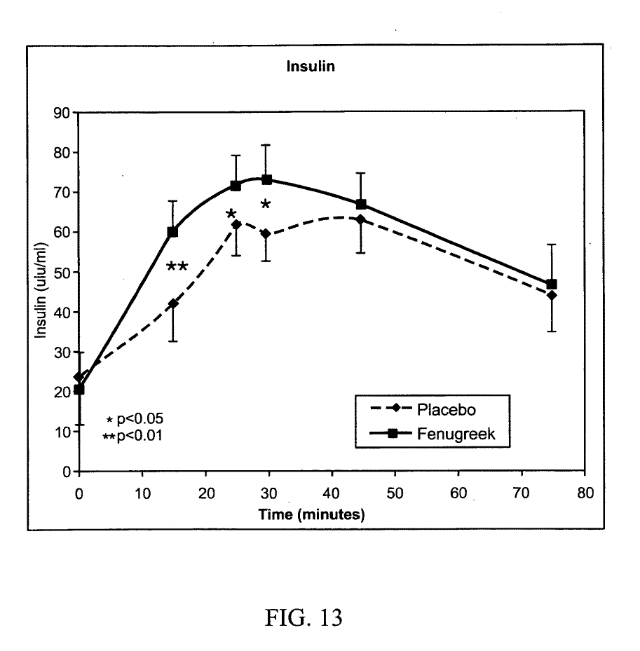

As shown in FIG. 13, a graph is shown of the insulin concentration plotted over the time course of the post-challenge sample times. As illustrated, the vertical axis (i.e., ordinate) references serum insulin activity concentrations measured in micro International Units per milliliter (μIU/mL) units. The horizontal axis (i.e., abscissa) references the sample times from time 0 (i.e., just before receiving glucose and the experimental challenge) to time 75 minutes. The confidence intervals for the plotted values are indicated by brackets. The data for the serum insulin response following placebo challenges are indicated by a broken line and the data for the serum insulin response following challenge with a novel composition of bio-active compounds derived, isolated, and/or extracted from fenugreek seeds are indicated by a solid line. Time points 25 and 30 minutes are marked with an asterisk (i.e., *) indicating significant difference (i.e., p<0.05) in insulin concentrations between challenges with PROMILIN™ and challenges with placebo. Time point 15 minutes is marked with a double asterisk (i.e., **) indicating highly significant difference (i.e., p<0.01) in insulin concentrations between challenge with PROMILIN™ and challenge with a placebo. These significant differences indicate a more abrupt increase in insulin concentration with the administration of PROMILIN™.

As illustrated in FIG. 13, the serum insulin concentrations were similar across trials for the 45 and 75 minute time points. The total area under the curve was significantly higher for the experimental trial for a number of time segments.

Referring now to FIG. 14, a bar graph illustrates the area under the curve for total insulin for time 0 to 45 minutes. The vertical axis (i.e., ordinate) referencing the insulin concentration, is measured in micro International Units per milliliter per minute (μIU/mL/min). The left bar illustrates the total area under the curve for the placebo trial and the right bar illustrates the total area under the curve for a trial with PROMILIN™. As further illustrated in FIG. 14, there is a most notable indication of the differences during the early stages of the trial which coincide with a rapid release of insulin during the initial 30 minutes post glucose ingestion.

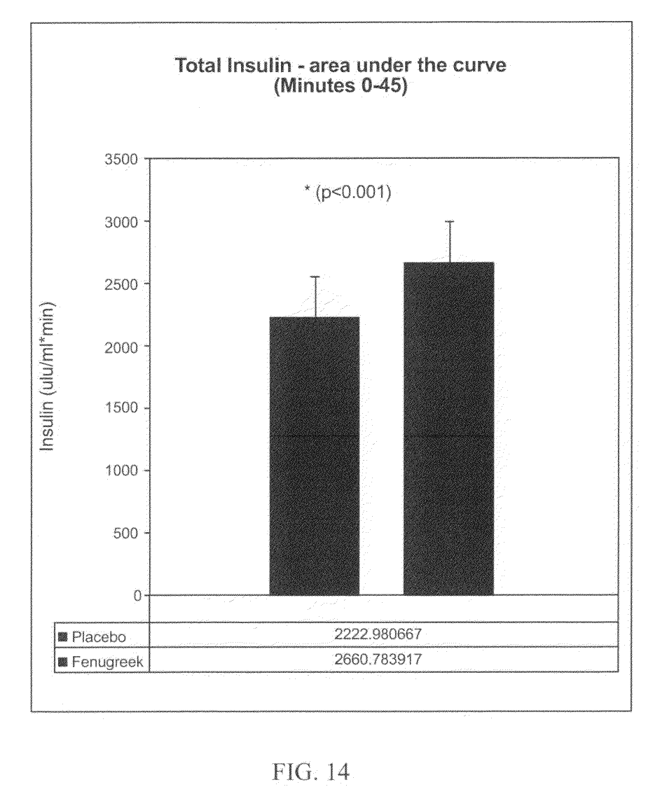

As shown in FIG. 15, a bar chart illustrates the percent (%) insulin concentration increase by time period following administration of PROMILIN™. The vertical axis (i.e., ordinate) references percent (%) insulin increase and the horizontal axis (i.e., abscissa) references time periods (measured in minutes following challenge with PROMILIN™. The overall total area under the curve (i.e., 0 to 75 minutes—as indicated by the hollow bar) was significantly higher for the experimental trial compared to placebo. As shown by the first three bars in FIG. 15 (as read from the left side), the experimental trial resulted in an insulin release pattern between from about fifteen percent (15%) to about twenty-one percent (21%) higher during the initial 30 minutes following oral glucose ingestion.

### Discussion

This example demonstrates the effects of an oral preparation containing a composition which includes the active amino acid potentiator, 4-hydroxyisoleucine, in promoting insulin release. The main findings indicate that, in combination with a large oral bolus of glucose (i.e., 1.8 g/kg BW), a novel composition of bio-active compounds derived, isolated, and/or extracted from fenugreek seeds (based on 2.0 mg PROMILIN™/kg of body weight) resulted in an abrupt and significantly greater insulin release during the initial 30 minutes following administration. Moreover, the total area under the curve (i.e., from about time 0 to about 75 minutes) for serum insulin was significantly higher with PROMILIN™ compared to placebo.

Serum insulin concentration was increased during the experimental trial by an average of 18.5% during the initial 15 minutes following the oral glucose bolus compared to the placebo. The insulin concentration was 14.2% higher on average during the experimental trial for the entire time 0 to 75 minute post-glucose ingestion.

In summary, the novel compositions of the present invention (referred to as PROMILIN™) appear to alter the physiological responses associated with a large oral bolus of glucose (i.e., 1.8 g/kg BW). These physiological responses may include, but are not limited to, the following: (1) an increase in gut absorption of glucose, as evidenced by the higher blood glucose at time 15 minutes during the experimental trial; (2) a stimulation of pancreatic beta cells (as evidenced by the rapid and sustained insulin concentrations during the experimental trial); and (3) enhanced glucose disposal (i.e., transference of something into a new place; e.g., as evidenced by the smaller total area under the curve for glucose in the experimental trial, indicating glucose is being transferred out of the blood and into tissues).

### EXAMPLE III

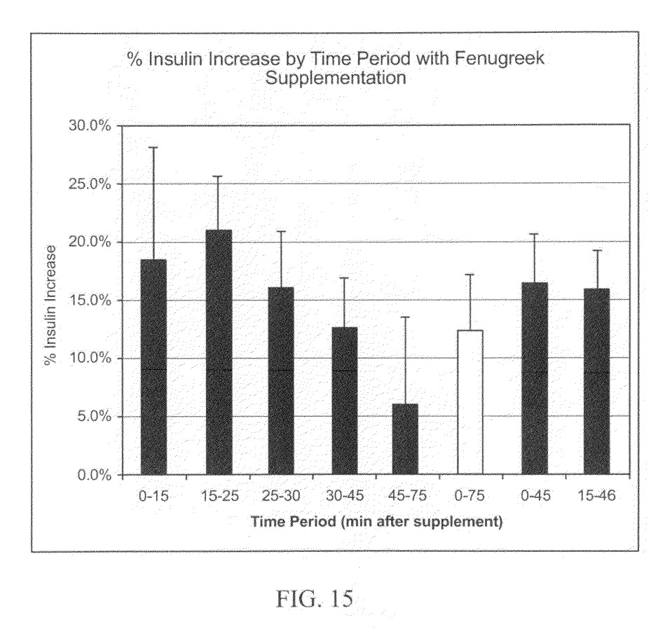

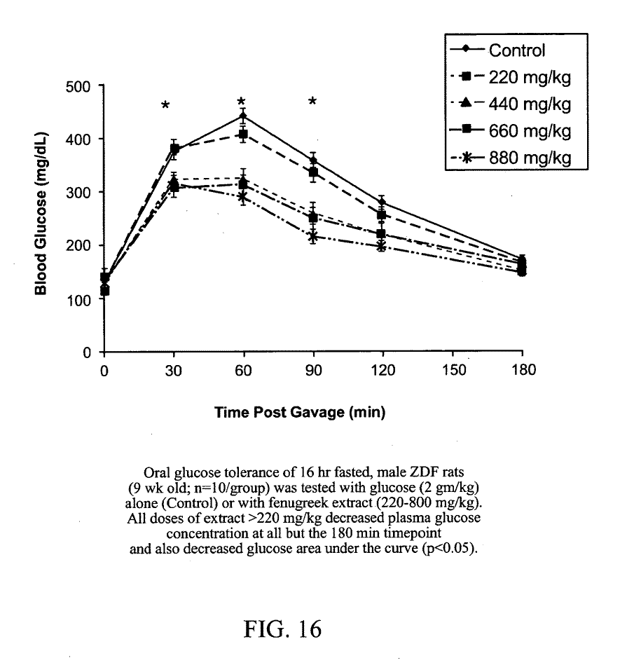

Referring generally to FIGS. 16-19, glucose tolerance tests were performed in strains of rats that were either insulin-resistant (Zucker) or diabetic (Zucker Diabetic Fatty, ZDF), which are strains selected as a models for human Type II diabetes mellitus. In experiment 1, glucose tolerance was assessed in 50 male ZDF/Gmi-fa rats, 9 weeks old, following a 16 hour fast. For oral glucose tolerances tests, the rats were divided into five groups of ten. The first group received a 60% glucose solution, 2 gm/kg body weight, by oral gavage, while the remaining four groups were given a gavage containing both glucose and one of four different dose levels of PROMILIN™, with ten animals per dose group.

PROMILIN™ was diluted in water to the appropriate dose level. Blood glucose concentrations were measured by taking blood samples from the tip of the tail, both before the gavage and at 30, 60, 90, 120, and 180 minute intervals post-gavage, using a MediSense Precision G blood glucose testing system. Intraperitoneal glucose tolerance tests were also performed. Twenty rats divided into two groups of ten were given an intraperitoneal gavage containing either 440 mg PROMILIN™/kg of body weight or plain water. Immediately after the gavage, both control (placebo) and the PROMILIN™rats were injected intraperitoneally with 1 mg/kg body weight of glucose solution. Blood samples were collected and tested in the same manner as for the oral test.

As seen in FIG. 16, in control rats, blood glucose increased four-fold to nearly 450 mg/dl by 60 minutes, whereas in rats given doses of 440 mg or more of fenugreek extract blood glucose rose only to around 300 mg/dl. In addition, the area under the respective curves is much smaller for the rats receiving PROMILIN™. As appreciated, it is generally believed by those skilled in the art that in evaluating deleterious effects of elevated blood sugar, the duration as well as the degree of elevation may be nearly as important as the peak level. Overall, oral administration of PROMILIN™ at doses of 220, 440, and 880 mg/kg of body weight decreased the positive adjusted area under the glucose curve compared to the control by 5.8% (NS or not significant), 49% (p<0.001) and 53% (p<0.001), respectively. Notably, the novel compositions of the present invention (referred to as PROMILIN™) improved glucose tolerance regardless of whether it was administered orally or by injection.

In addition, insulin levels were determined in the blood samples. Plasma insulin concentration was measured with a commercially available test (e.g., the ALPCO Ultrasensitive Rat Insulin ELISA kit). Oral administration of PROMILIN™ did elevate insulin in plasma, but this effect was short-lived compared to the effects on glucose levels (see FIG. 17; Note: the 90 minute time point was lost). Thus, it can be concluded that PROMILIN™ improved glucose tolerance without significantly increasing plasma insulin concentration.

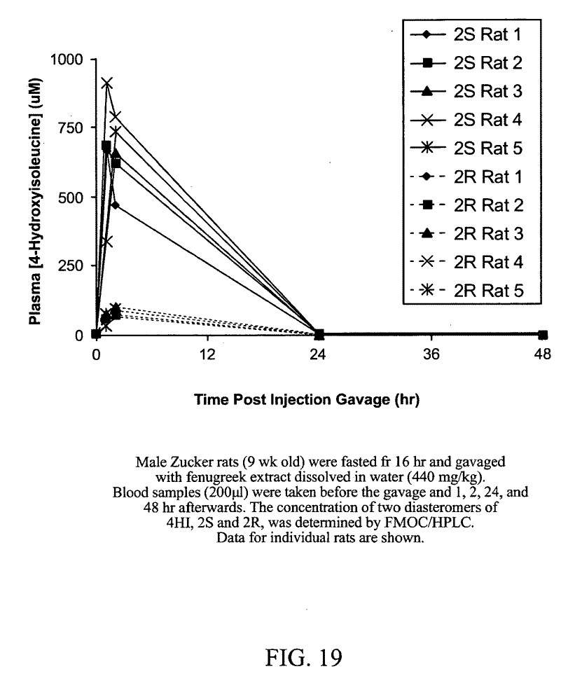

In a third test, male Zucker rats (five animals, 9 weeks old) were fasted for 16 hours and orally gavaged with 440 mg/kg of PROMILIN™ dissolved in water. Blood samples (200 μl) were taken from the tip of the tail before the gavage and injection and at intervals after; the PROMILIN™ samples were analyzed by HPLC for the concentrations of two diastereomers (2S,3R,4S and 2R,3R,4S) of 4-hydroxyisoleucine (collectively referred to as 4-OH-Ile). Blood 4-OH-Ile was measured by reverse-phase HPLC of the FMOC derivation of 4-OH-Ile with fluorescence detection, quantified against spiked samples. Oral administration of PROMILIN™ transiently elevated the plasma concentration of 4-OH-Ile (see FIG. 19).

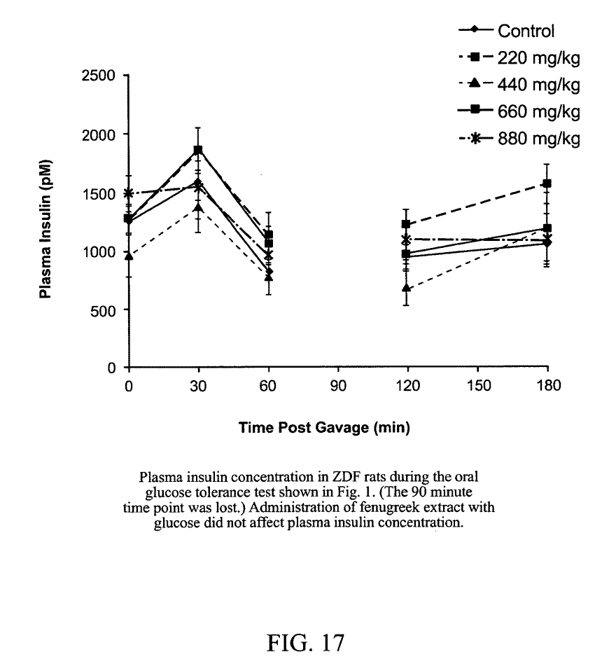

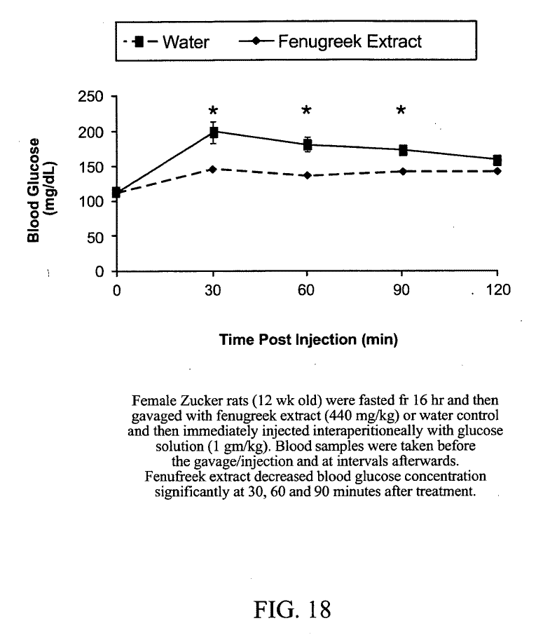

In experiment 2, five 12-week-old female Zucker fa/fa rats were fasted for 16 hours, gavaged with 440 mg PROMILIN™/kg of body weight or water control, and then immediately injected intraperitoneally with glucose (1 gm/kg). Tail tip blood samples were taken before the gavage/injection and at 30 minute intervals for 2 hours. PROMILIN™ decreased blood glucose concentration significantly at 30, 60, and 90 minutes post-treatment (see FIG. 18).

From the results set forth in FIGS. 16-19 and other results from the two experiments conducted, it can be supportably concluded that PROMILIN™ improves glucose tolerance in diabetic and pre-diabetic rats. The effect is apparent even when glucose is given by injection, suggesting that the effect of the administration of the compositions of the present invention (referred to herein as PROMILIN™) is outside the gastrointestinal tract. Oral administration of PROMILIN™ also resulted in a transient increase in the blood level of 4-OH-Ile. Further, while it was previously known that 4-OH-Ile could stimulate insulin secretion, the present studies show that a considerable part of the glucose tolerance improving effects of PROMILIN™ cannot be ascribed to an increase in plasma insulin levels.

### EXAMPLE IV

### Prediabetic Human Subjects

A randomized, double-blind, placebo-controlled study was performed to determine the efficacy of three different doses of PROMILIN™ versus placebo in prediabetic human patients with Metabolic Syndrome X who met certain criteria. PROMILIN™ used in this study involved the particular embodiments of the 4-OH-Ile containing compositions which are described in Examples V-VIII hereafter. The use of the embodiments of compositions of the present invention as described in Examples V-VIII are not intended, however, to be a limitation on the components of compositions that may prove useful for improving glycemic control in humans.

In addition to the human clinical portion of the study, preclinical trials were conducted in rats to assess if the 4-OH-Ile in PROMILIN™ is orally bioavailable to rats and if it is excreted in their urine. The preclinical studies demonstrated that 4-OH-Ile was bioavailable in rats and in particular that both diastereomers of the 4-OH-Ile in the PROMILIN™ administered were absorbed from the gut into the circulation within 15 minutes of oral gavage. Coadministration of glucose and PROMILIN™ slowed the appearance of 4-OH-Ile in the circulation. Moreover, both isomers were shown to be excreted in the urine.

For the human study, subjects were recruited from Western Montana to participate in this randomized, double-blind, placebo-controlled study of the effects of PROMILIN™ relative to improving glycemic control in humans. The original pool of volunteers were screened for fasting blood glucose levels and a total of 35 subjects who had a diagnosis of Syndrome X and met certain specific criteria for inclusion and exclusion were enrolled in the study. These were randomized into four treatment subgroups for the study. Criteria for inclusion in the study included the following: subjects had to (1) be over 18 years old; and (2) have a diagnosis of Syndrome X, exhibiting at least three of the following six indicators: (I) fasting blood glucose>110 mg/dL but <126 mg/dL in at least two tests on different visits; (ii) blood pressure≧130/85; (iii) HDL (high-density lipoprotein)<40 mg/dL for males or <50 mg/dL for women; (iv) a triglyceride level≧150 mg/dL; (v) abdominal obesity with a waistline of >40 inches for men or >35 inches for women; (vi) a first-degree relative with a diagnosis of diabetes mellitus.

In addition, the study excluded participants with any of the following characteristics: (a) a confirmed diagnosis of diabetes Type I or Type II; (b) individuals that were pregnant or breast-feeding; \textcircled{c} individuals using insulin or other diabetes medications (i.e., sulfonylureas, biguanides, glycosidase inhibitors, glitazones, or meglitinides); (d) individuals using anticoagulants, antiplatelet drugs, or non-steroidal anti-inflammatory agents (i.e., NSAIDS, e.g. Motrin, Advil, Aleve, or the like); (e) individuals using herbal supplements or products tending to cause hypoglycemia or anticoagulation, including for example those containing ginger, gotu kola, Dong Quai, and garlic, or other anti-diabetic herbal formulas; (f) individuals with renal function impairment (serum creatinine>1.5 mg/dL for men and >1.2 mg/dL for women); (g) the existence of any other condition that might cause elevated blood glucose tests, including secondary pancreatic diseases or endocrinopathies; individuals taking any of the following: beta adrenergic blockers, glucocorticoids, certain diuretics, calcium channel blockers, rifampin, lithium salts, niacin, glycerol, asparaginase, sympathomimetics, diazoxide, pentamidine isoethionate; or phenyloin sodium; (h) chronic alcohol consumption; (j) a diagnosis of hypothyroidism or polycystic ovary disease; (k) impaired liver function with liver function tests three times above normal levels; and (l) individuals currently enrolled in another investigational trial, or those whose health status is not stable due to other chronic conditions or having a disease state which the study product might worsen or interfere with current treatment.

The 35 enrolled participants were randomly assigned to one of four subgroups A, B, C, and D according to placebo or PROMILIN™ treatment level. There were thus eight to nine individuals within each group. Three dosage levels of PROMILIN™ were studied: 200 mg, 400 mg, and 800 mg. Assuming an average male human as having a body weight of 80 kg (175 lbs) and considering the analyses of Examples V-VIII indicating that the described embodiments of PROMILIN™ comprises roughly 25% by weight of 4-OH-Ile, the three doses tested (200 mg, 400 mg, and 800 mg) amount to roughly 0.5 mg/kg body weight, 1.0 mg/kg body weight, and 2.0 mg/kg body weight of 4-OH-Ile, respectively. The placebo capsules contained 200 mg microcrystalline cellulose and each treatment capsule contained cellulose blended with 1.0% PROMILIN™ providing 200 mg PROMILIN™. Subjects were given a total of four capsules, comprising either placebo capsules only (Group A); three placebo plus one capsule containing PROMILIN™ (Group B); two placebo plus two capsules containing PROMILIN™ (Group C); or four capsules containing PROMILIN™ (Group D).

The study was conducted in two trials separated by approximately a week of time. In the baseline trial, fasting blood glucose levels were taken after overnight fasting by the subjects, and a glucose tolerance test performed, without dosing. In the PROMILIN™ test trial, subjects again fasted overnight before coming to the laboratory, and an initial fasting blood glucose level was determined upon arrival. At this second trial, subjects in the four treatment groups were given either a placebo (Group A) or an oral dose of PROMILIN™ (Groups B, C, and D received doses of 200 mg, 400 mg, and 800 mg, respectively). One hour following administration of the placebo or PROMILIN™ test dose, the fasting blood glucose levels were again measured. Once the second set of fasting blood glucose levels was taken, a glucose tolerance test was initiated in all four groups by orally administering a glucose challenge of about 75 grams of glucose. Blood glucose levels were then determined at one and two hours post-challenge. From the post-challenge measurements of blood glucose levels versus time, the adjusted peak blood glucose (APBG) level and the area under the curve (AUC) were determined. The placebo and PROMILIN™ test doses were administered and the blood glucose measurements made in a coded double-blind manner, and the results were later decoded for statistical analysis.

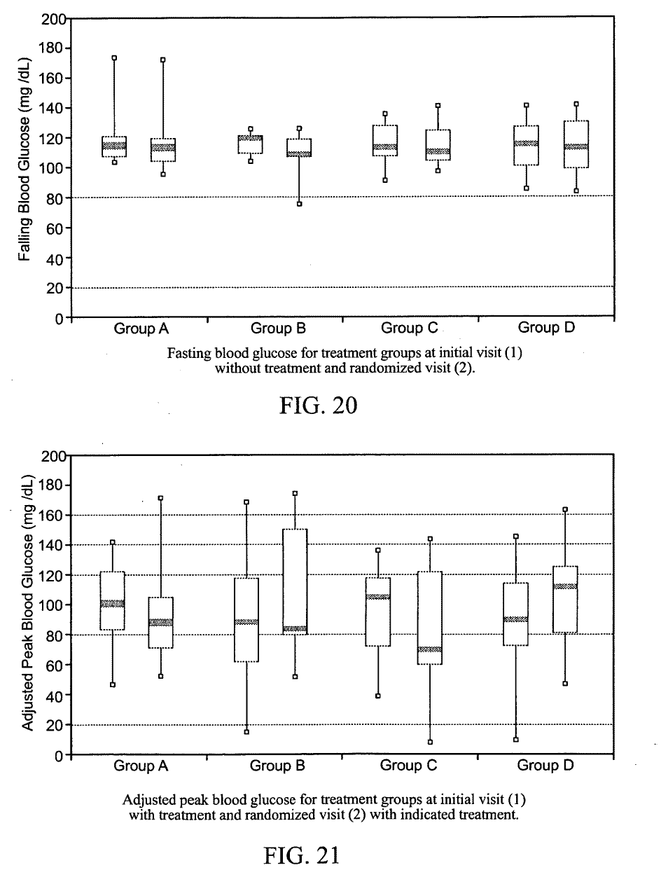

The results of the test are depicted in the bar graphs of FIGS. 20-22, where a pair of bars is shown for each of the four treatment Groups A-D (administering placebo, 200 mg, 400 mg, and 800 mg, respectively). Various statistical analyses were performed to establish the level of baseline variation among the four groups (without any treatment); while there was some variability, there were no significant overall group differences.

Referring specifically to FIG. 20, the left-hand most bar in each group represents the baseline fasting blood glucose level and the right-hand bar represents the blood glucose level following administration of the placebo or PROMILIN™ dose. There was a slight drop in the 200 mg treatment group, but otherwise PROMILIN™showed no effect on fasting blood glucose level.

Turning now to FIG. 21, this bar graph depicts blood glucose levels measured in the glucose tolerance test administered to the four treatment groups. The peak adjusted blood glucose (PABG) level was the blood glucose level measured at one hour following administration of the glucose bolus, minus the baseline fasting blood glucose level. The left bar in each group represents the baseline (no treatment given other than glucose challenge), while the right bar represents the dose trial measurements for the four groups. Note there is some variation in baseline PABG among the four groups, which is likely due to the small sample size and the variable constellation of symptoms used to classify participants as having Syndrome X. Three doses of PROMILIN™ were compared to the baseline data.

Discussing the results of this study, there was variability among the individuals within each of the four subject Groups A-D in fasting glucose blood levels and other measurements, as evidenced by the two-fold range of measurements within each group. This variability is believed due in part to the small sample numbers (e.g., 7 to 10 individuals per treatment group). Fasting blood levels were fairly consistent among the four groups in both the baseline and the dose trials (see FIG. 20), averaging about 115 mg/dL (milligrams per deciliter). In the corresponding Figures, the shaded part of the bar is the average for the group, the open box represents the confidence levels p<0.01 of that average, and the extended bar indicates the range of measurements for the treatment group.

The peak blood glucose level following administration of the glucose challenge in a glucose tolerance test may be seen as reflective of the degree of glycemic control of an individual animal or human. FIG. 21 depicts adjusted peak blood glucose levels for the four treatment Groups A-D in two sets of glucose tolerance tests in mg/dL (mg per deciliter). As can be seen from FIG. 21, there is some variation in peak blood glucose among individuals and between the groups when no PROMILIN™ at all was administered (left-most bar for each pair). Looking closely, it can be seen that peak adjusted blood glucose levels (PABG levels) were 100 mg/dL (range 45-140) in the first test and 85 mg/dL (range 50-170) in the second test, respectively, for Group A. For Group B, PABG levels without PROMILIN™ (left bar) were 88 mg/dL (range 15-170), and with 200 mg PROMILIN™ were 82 mg/dL (range 48-178). For Group C, PABG levels without PROMILIN™ were 110 mg/dL (range 40-138), and with 400 mg PROMILIN™ were 68 mg/dL (range 10-143). For Group D, PABG levels in the first test (no PROMILIN™) were 92 mg/dL (range 10-145), and in the second test (800 mg PROMILIN™) were 110 mg/dL (range 45-170) (referring to FIG. 21). From these data, it can be seen that the 400 mg dose of PROMILIN™ (administered to Group C) reduced the average PABG from about 110 mg/dL to about 68 mg/dL.

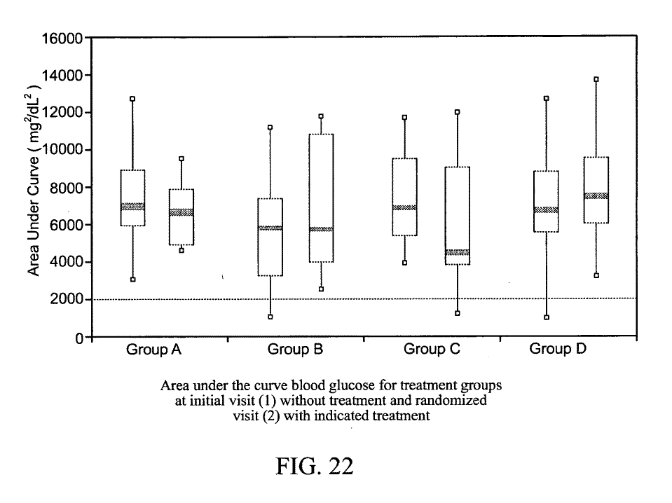

As previously mentioned, the area under the curve of blood glucose vs. time following administration of a glucose challenge to a fasting individual is another measure used by those skilled in the art to assess glucose tolerance and glycemic control in humans and animals. In FIG. 22, the area under the curve (AUC) represents the integrated area under the glucose tolerance curves from time zero to time equals 2 hours following the administration of the glucose challenge, either with or without a dose of PROMILIN™. As can be seen, there were some differences between the average and the distribution of AUC for the four treatment groups in the first set of tests (glucose challenge only; left-most bar for each treatment Group A-D). In the second set of tests, P PROMILIN™ was administered together with glucose at dose of 0 (Group A), 200 mg (Group B), 400 mg (Group C), or 800 mg (Group D). As can be seen in FIG. 22, the 400 mg dose of PROMILIN™ (Group C) resulted in a distinct lowering of the average AUC, from 6800 to 4300, while there was essentially no difference between the baseline and PROMILIN™ trial tests for the other three groups.

Paired t-tests were conducted to describe within-treatment-group changes in the variables in the baseline vs. PROMILIN™ trial tests. For group C, which received the 400 mg dose in the PROMILIN™ trial, there was a 9.8% reduction in the peak adjusted blood glucose (PABG) level (p=0.028), and an 18.3% reduction in the AUC (p=0.025.) These results demonstrate that the 400 mg dose of PROMILIN™ administered concurrently with a caloric intake (the glucose challenge) was effective to improve glycemic control in this group of Syndrome X subjects.

The 200 mg dose of PROMILIN™ may have been too low to show an effect. It is not clear why the higher dose of 800 mg of PROMILIN™ did not produce any effect in this set of tests, but this may be related to the small sample sizes and/or to the heterogeneity of Syndrome X. As known to those skilled in the art, Syndrome X is used to describe individuals having a constellation of symptoms thought to reflect a pre-diabetic condition, including but not limited to excess body fat, particularly abdominal fat; abnormally high fasting blood glucose levels; and high post-challenge glucose levels in glucose tolerance tests.

Overall, the study results indicated that PROMILIN™ was well tolerated at all dose levels; no adverse events were reported among the study participants. The results also demonstrated that at a dose of 400 mg of PROMILIN™, corresponding roughly to 1 mg/kg of 4-OH-Ile, there was an improvement in glucose tolerance as reflected in lower peak adjusted blood glucose level and in a lower AUC (see FIGS. 21 and 22). This data did not show a linear dose response, i.e., the higher (800 mg) dose of PROMILIN™ did not produce a greater improvement in glucose tolerance test results. However, there are several possible reasons for this. First, the sample size is relatively small (eight to ten individuals per treatment group). Second, Syndrome X is not a fully defined disorder and subjects diagnosed as having Syndrome X vary somewhat in the precise constellation of symptoms (see study inclusion criteria, above). Further, non-linear dose-response curves are known to those skilled in the art of pharmacology for some compounds or compositions, and such cannot be ruled out for PROMILIN™. Also, it is noted that some of the other data described in relation to Examples I-III herein suggest a non-linear dose-response for the composition of the invention.

Consistent with the foregoing, Examples I-IV focus on testing of certain methods of using various embodiments of PROMILIN™ for improving glucose tolerance in mammals having disordered carbohydrate metabolism leading to excess blood sugar levels and improving glycemic control in humans. As appreciated, with plant-derived compositions, to achieve the best results with the method, it is highly desirable to have means quantifying the chemical content of key components such as 4-OH-Ile. Accordingly, the inventors developed a quality control process for analyzing the composition prepared from fenugreek seeds using high pressure liquid chromatography (HPLC) This process comprises assembling an HPLC apparatus including a fluorescence detector and programmable autosampler, and may be utilized in a methods validation program. The chromatography column may be a Zorbax stable bond SB-C18 chromatography column (4.6*150 mm, 5 μm). Desirably, the HPLC apparatus may includes an analytical balance, accurate to 0.1 mg, an ultrasonic bath, a volumetric flask, a two liter vacuum filtration glassware with 0.2 μm membrane, variable volumetric pipettes, and a magnetic stirrer and stir bars.

The reagents used in the embodiments of the quality control process of a methods validation program may include, for example: (1) methanol (HPLC grade), (2) acetonitrile (HPLC grade), (3) sodium acetate trihydrate (AR grade), (4) triethylamine (AR grade), (5) glacial acetic acid (AR grade), (6) tetrafuran (AR grade), (7) OPA reagent (Agilent Co. Part No. 5061-3335, containing o-phthaldialdehyde and 3-mercaptopropionic acid in borate buffer), (8) a reference standard of 4-hydroxyisoleucine (obtained from British Agricultural Lab), and (9) de-ionized water.

One embodiment of a quality control process of the present invention comprising a methods validation program may include a mobile phase step, a standard preparation step, and a sample preparation step. In the mobile phase step, buffer A, buffer B, and a filter/degas step may be utilized. Buffer A may be prepared in a one-liter beaker, wherein 1.36 g of sodium acetate trihydrate may be dissolved in 500 mL water. This combination may be stirred until thoroughly dissolved. Ninety (90) μL of triethylamine may be added and mixed. The pH may be adjusted to about 7.2 with between from about one percent (1%) to about two percent (2%) of acetic acid solution. 1.5 mL of tetrafuran may then be added and mixed. The final mixture may be labeled—“buffer A.”

Buffer B may be formed in accordance with the following procedure. In a beaker, one point three-six (1.36) g sodium acetate trihydrate may be dissolved in 100 mL of water. This combination may be stirred until thoroughly dissolved. The pH may be adjusted to 7.2 with between from about one percent (1%) to about two percent (2%) acetic acid solution. 200 mL of methanol and 200 mL of acetonitrile may then be added to the beaker and mixed well. The final mixture may be labeled—“buffer B.” Preferably, the buffers may be filtered and degassed using a vacuum and 0.2 μm membrane.

In one embodiment, a quality control process of the present invention may include a methods validation program comprising the use of a reference compound to provide a known standard in the assay. In one embodiment, 10 mg of a reference compound is accurately weighed out and accurately weighing about 10 mg of a reference compound and placing the compound into a 50 mL volumetric flask. The reference compound may be dissolved using about 30 mL deionized water, and then undergoes sonication for approximately 10 minutes. The flask is preferably allowed to cool to room temperature; the solution may then be diluted with water to a specific concentration and mixed well. The standard preparation may then be sealed with a parafilm and stored under refrigeration until needed.

In one embodiment, a quality control analysis process on a sample may be performed as follows: (1) a methods validation program sample may include a preparation step wherein about 25 mg of PROMILIN™ is accurately weighed with precision and dissolved with about 30 mL deionized water in a 50 mL volumetric flask, and sonicated undergoing sonicate for approximately 10 minutes. The flask is preferably allowed to cool to room temperature and then the solution may be diluted with water to a desired concentration and mixed well. Optionally, the sample preparation may be filtered prior to being injected into an HPLC apparatus.

Chromatographic conditions for an embodiment of a quality control process may comprise a Zorbax stable bond SB-C18 column operating at a column temperature of 30° Celsius (C), and an EX 340 nM, EM 450 chromatographic detector. The following gradients and injection program may be utilized:

Gradient:

| Time (min) | % A | % B | F (ml/min) |
|---|---|---|---|
| 0.00 | 100 | 0 | 1.0 |
| 17.0 | 50 | 50 | 1.0 |
| 20.0 | 0 | 100 | 1.0 |
| 20.1 | 0 | 100 | 1.0 |
| 24.0 | 100 | 0 | 1.0 |
| 35.0 | 100 | 0 | 1.0 |

Injection program:

| Row | Action |
|---|---|
| 1 | Draw 5.0 μL from vial 1 (buffer) |
| 2 | Draw 1.0 μL from vial 2 (sample) |
| 3 | Mix 6.0 μL in air, max. speed, 6 times |
| 4 | Submerge injector tip in vial 11 (wash |
| | vial) |
| 5 | Draw 1.0 μL from vial 3 (OPA reagent) |
| 6 | Mix 7.0 μL in air, max. speed, 6 times |
| 7 | Submerge injector tip in vial 11 (wash |
| | vial) |
| 8 | Inject |

Analysis of the results of the quality control test may be performed by examining the spectrum including the peak height and area under the curve of identified peaks (peak area). Under proper conditions, peak height correlates with the amount of the test sample; the correlation must be established using known amounts of the reference standard. HPLC is known to be nonlinear in some concentration ranges of the analyte, that is the peak height will not accurately correspond to concentration, so it is highly desirable to determine the concentration range in which the relationship of peak size to analyte concentration is linear. To determine the region of linearity, the inventors prepared a standard preparations of 4-OH-Ile at different known concentrations and assayed them as directed in a quality control process of the present invention. One such linearity analysis was performed and the results indicated that there was good linearity in the range from 0.09 mg/ml to 0.27 mg/ml 4-OH-Ile, with a correlation coefficient of R>0.99950. Thus, this embodiment of a quality control protocol of the present invention is be suitable for analysis of the composition of the invention.

Precision of the quality control analysis may be assessed by performing multiple separated tests on a single test sample. Three trials were made with three different known test samples; in each trial, six separate HPLC analyses were performed on the same sample. The relative standard deviations for the three trials were, respectively, 0.66%, 0.87%, and 1.0%. Taken together, the relative standard deviation for these analyses was <three percent (3%). Thus, the embodiment of a quality control analysis as contemplated by the present invention delivered good precision for the sample.

Reproducibility of the quality control process was assessed by testing a single known sample with multiple HPLC assays on consecutive days. The results showed a high degree of reproducibility, with a relative standard deviation RSD of <three percent (3%).

A quality control protocol according to the above-described embodiment was conducted and analyzed for recovery and accuracy using spiked and recovered sample analyte and spiked and recovered standard analyte. The following results were observed:

4-OH-Ile Recovery

| | | Spiked | Recovered | | |
|---|---|---|---|---|---|
| | Sample | (4-OH | (4-OH | Recovery | Overall |
| | Spiked | ILE) | ILE) | (4-OH ILE) | Average |
| | (mg) | (mg) | (mg) | (%) | (%) |
| FSE2060052 + | 10.8 | 5.34 | 5.23 | 98.0 | |
| FSE02G31-32 | | | | | |
| FSE2060052 + | 22.1 | 10.92 | 10.68 | 97.8 | |
| FSE02G31-32 | | | | | |
| FSE2060052 + | 33.9 | 16.75 | 16.41 | 98.0 | |
| FSE02G31-32 | | | | | |
| FSE20020402 + | 10.2 | 5.04 | 4.98 | 98.7 | |
| FSE02G31-32 | | | | | |
| FSE20020402 + | 21.6 | 10.67 | 10.37 | 97.2 | |
| FSE02G31-32 | | | | | |
| FSE20020402 + | 30.8 | 15.22 | 14.84 | 97.6 | 97.9 |
| FSE02G31-32 | | | | | |

These foregoing data demonstrate that one embodiment of the quality control (QC) process has good accuracy. Thus, this embodiment of the QC process is useful to provide precise information as to the content of 4-hydroxyisoleucine in different lots of PROMILIN™ of the present invention.

### EXAMPLE V

Lot No. 2090769 was analyzed using the HPLC-based quality control process, as previously described, and was found to contain an embodiment of a composition of bio-active compounds derived, isolated, and/or extracted from fenugreek seeds of the present invention resulting in the following composition (referred to herein as an embodiment of PROMILIN™):

| Measurement | % (w/w) |
|---|---|
| Protein | 42.52 |
| Oil Content | 0.20 |
| Ash | 3.19 |
| Moisture | 13.10 |
| Soluble Fiber | 2.30 |
| Insoluble Fiber | 0.90 |
| Amino Acids | |
| Arginine | 1.92 |
| Aspartate | 1.94 |
| Threonine | 0.43 |
| Serine | 0.32 |
| Glutamate | 3.23 |
| Proline | 0.41 |
| Glycine | 1.03 |
| Alanine | 1.17 |
| Cysteine | 0.08 |
| Valine | 0.25 |
| Methionine | 0.29 |
| Isoleucine | 0.26 |
| Leucine | 0.28 |
| Tryptophan | 0.14 |
| Phenylalanine | 0.73 |
| Lysine | 0.22 |
| Histidine | 0.29 |
| Tyrosine | 0.03 |
| 4-hydroxyisoleucine | 24.50 |
| Total Amino Acids | 37.79 |

As illustrated hereinabove, the embodiment of PROMILIN™ consists of about forty-three percent (43%) protein, about 0.2% oil, about 3.19% ash, about 13.10% moisture, about 2.30% insoluble fiber, about 0.90% soluble fiber and about thirty-eight percent (38%) free amino acids, including about twenty-five percent (25%) 4-OH-Ile and various quantities of the following amino acids: arginine, aspartate, threonine, serine, glutamate, proline, glycine, alanine, cysteine, valine, methionine, isoleucine, leucine, tryptophan, phenylalanine, lysine, histidine, and tyrosine.

Since the embodiments of PROMILIN™ described in the present invention include 4-OH-Ile and one or more amino acids, it will be readily appreciated that further embodiments of PROMILIN™ may contain 4-OH-Ile and one or more amino acids selected from the group consisting of arginine, aspartate, threonine, serine, glutamate, proline, glycine, alanine, cysteine, valine, methionine, isoleucine, leucine, tryptophan, phenylalanine, lysine, histidine, and tyrosine. It is intended, therefore, that the Examples provided herein be viewed as exemplary of the principles of the present invention, and not as restrictive to a particular structure or method for implementing those principles.

### EXAMPLE VI

Lot No. 2121492 was analyzed using the HPLC-based quality control process, as previously described, and was found to contain an embodiment of a composition of bio-active compounds derived, isolated, and/or extracted from fenugreek seeds of the present invention resulting in the following composition:

| Measurement | % (w/w) |
|---|---|
| Protein | 52.43 |
| Oil Content | 0.07 |
| Ash | 1.59 |
| Moisture | 8.42 |
| Soluble Fiber | 1.80 |
| Insoluble Fiber | 0.20 |
| Amino Acids | |
| Arginine | 1.46 |
| Aspartate | 1.51 |
| Threonine | 0.34 |
| Serine | 0.12 |
| Glutamate | 3.05 |
| Proline | 0.37 |
| Glycine | 0.96 |
| Alanine | 1.31 |
| Cysteine | 0.07 |
| Valine | 0.35 |
| Methionine | 0.24 |
| Isoleucine | 0.23 |
| Leucine | 0.18 |
| Tryptophan | 0.02 |
| Phenylalanine | 0.33 |
| Lysine | 0.19 |
| Histidine | 0.29 |
| Tyrosine | 0.07 |
| 4-hydroxyisoleucine | 24.40 |
| Total Amino Acids | 35.49 |

As illustrated hereinabove, the embodiment of PROMILIN™ consists of about fifty-two percent (52%) protein, about 0.07% oil, about 1.59% ash, about 8.42% moisture, about 1.80% insoluble fiber, about 0.20% soluble fiber and about thirty-five percent (35%) free amino acids, including about 24% 4-OH-Ile and various quantities of the following amino acids: arginine, aspartate, threonine, serine, glutamate, proline, glycine, alanine, cysteine, valine, methionine, isoleucine, leucine, tryptophan, phenylalanine, lysine, histidine, and tyrosine.

Since the embodiments of PROMILIN™ described in the present invention include 4-OH-Ile and one or more amino acids, it will be readily appreciated that further embodiments of PROMILIN™ may contain 4-OH-Ile and one or more amino acids selected from the group consisting of arginine, aspartate, threonine, serine, glutamate, proline, glycine, alanine, cysteine, valine, methionine, isoleucine, leucine, tryptophan, phenylalanine, lysine, histidine, and tyrosine. It is intended, therefore, that the Examples provided herein be viewed as exemplary of the principles of the present invention, and not as restrictive to a particular structure or method for implementing those principles.

### EXAMPLE VII

Lot No. 2101114 was analyzed using the HPLC-based quality control process, as previously described, and was found to contain an embodiment of a composition of bio-active compounds derived, isolated, and/or extracted from fenugreek seeds of the present invention resulting in the following composition (referred to herein as an embodiment of PROMILIN™):

| Measurement | |
|---|---|
| Amino Acids | % (w/w) |
| Arginine | 1.09 |
| Aspartate | 1.82 |
| Threonine | 0.41 |
| Serine | 1.71 |
| Glutamate | 3.09 |
| Proline | 0.20 |
| Glycine | 0.94 |
| Alanine | 1.48 |
| Cysteine | 0.79 |
| Valine | 0.46 |
| Methionine | 0.15 |
| Isoleucine | 0.21 |
| Leucine | 0.20 |
| Tryptophan | 0.81 |
| Phenylalanine | 0.73 |
| Lysine | 0.17 |
| Histidine | 0.16 |
| Ornithine | 0.06 |
| Gamma-aminobutyrate | 0.34 |
| 4-hydroxyisoleucine | 26.00 |
| Total Amino Acids | 40.82 |

As appreciated by those skilled in the art, the presently preferred embodiment of the present invention consists of about forty-one percent (41%) free amino acids, including about twenty-six percent (26%) 4-OH-Ile and various quantities of the following amino acids: arginine, aspartate, threonine, serine, glutamate, proline, glycine, alanine, cysteine, valine, methionine, isoleucine, leucine, tryptophan, phenylalanine, lysine, histidine, ornithine, and gamma-aminobutyrate.

Since the embodiments of PROMILIN™ described in the present invention include 4-OH-Ile and one or more amino acids, it will be readily appreciated that further embodiments of PROMILIN™ may contain 4-OH-Ile and one or more amino acids selected from the group consisting of arginine, aspartate, threonine, serine, glutamate, proline, glycine, alanine, cysteine, valine, methionine, isoleucine, leucine, tryptophan, phenylalanine, lysine, histidine, and tyrosine. It is intended, therefore, that the Examples provided herein be viewed as exemplary of the principles of the present invention, and not as restrictive to a particular structure or method for implementing those principles.

### EXAMPLE VIII

Lot No. 2101055 was analyzed using the HPLC-based quality control process, as previously described, and was found to contain another presently preferred embodiment of a composition of bio-active compounds derived, isolated, and/or extracted from fenugreek seeds of the present invention resulting in the following composition (referred to herein as an embodiment of PROMILIN™):

| Measurement | |
|---|---|
| Amino Acids | % (w/w) |
| Arginine | 0.90 |
| Aspartate | 1.49 |
| Threonine | 0.35 |
| Serine | 4.44 |
| Glutamate | 2.47 |
| Glycine | 0.81 |
| Alanine | 1.22 |
| Cysteine | 0.67 |
| Valine | 0.41 |
| Methionine | 0.20 |
| Isoleucine | 0.20 |
| Leucine | 0.17 |
| Tryptophan | 0.69 |
| Phenylalanine | 0.61 |
| Lysine | 0.13 |
| Histidine | 0.14 |
| Ornithine | 0.04 |
| Gamma-aminobutyrate | 0.29 |
| 4-hydroxyisoleucine | 23.26 |
| Total Amino Acids | 38.49 |

As appreciated by those skilled in the art, the embodiment of PROMILIN™consists of about thirty-nine percent (39%) free amino acids, including about twenty-three percent (23%) 4-OH-Ile and various quantities of the following amino acids: arginine, aspartate, threonine, serine, glutamate, glycine, alanine, cysteine, valine, methionine, isoleucine, leucine, tryptophan, phenylalanine, lysine, histidine, ornithine, and gamma-aminobutyrate.

Since the embodiments of PROMILIN™ described in the present invention include 4-OH-Ile and one or more amino acids, it will be readily appreciated that further embodiments of PROMILIN™ may contain 4-OH-Ile and one or more amino acids selected from the group consisting of arginine, aspartate, threonine, serine, glutamate, proline, glycine, alanine, cysteine, valine, methionine, isoleucine, leucine, tryptophan, phenylalanine, lysine, histidine, and tyrosine. It is intended, therefore, that the Examples provided herein be viewed as exemplary of the principles of the present invention, and not as restrictive to a particular structure or method for implementing those principles.

### EXAMPLE IX

Lot No. 2090898 was analyzed using the HPLC-based quality control process, as previously described, and was found to contain another presently preferred embodiment of a composition of bio-active compounds derived, isolated, and/or extracted from fenugreek seeds of the present invention resulting in the following composition (referred to herein as an embodiment of PROMILIN™):

| Measurement | |
|---|---|
| Amino Acids | % (w/w) |
| Arginine | 0.81 |
| Aspartate | 1.27 |
| Threonine | 0.23 |
| Serine | 0.87 |
| Glutamate | 1.96 |
| Glycine | 0.67 |
| Alanine | 1.17 |
| Cysteine | 0.74 |
| Valine | 0.36 |
| Methionine | 0.10 |
| Isoleucine | 0.22 |
| Leucine | 0.21 |
| Phenylalanine | 0.56 |
| Ornithine | 0.08 |
| Lysine | 0.13 |
| Histidine | 0.10 |
| Tyrosine | 0.42 |
| 4-hydroxyisoleucine | 24.11 |
| Total Amino Acids | 34.01 |

As appreciated by those skilled in the art, the embodiment of PROMILIN™ consists of about 34% free amino acids, including about twenty-four percent (24%) 4-OH-Ile and quantities of the following amino acids: arginine, aspartate, threonine, serine, glutamate, glycine, alanine, cysteine, valine, methionine, isoleucine, leucine, phenylalanine, ornithine, lysine, histidine, and tyrosine.

Since the embodiments of PROMILIN™ described in the present invention include 4-OH-Ile and one or more amino acids, it will be readily appreciated that further embodiments of PROMILIN™ may contain 4-OH-Ile and one or more amino acids selected from the group consisting of arginine, aspartate, threonine, serine, glutamate, proline, glycine, alanine, cysteine, valine, methionine, isoleucine, leucine, tryptophan, phenylalanine, lysine, histidine, and tyrosine. It is intended, therefore, that the Examples provided herein be viewed as exemplary of the principles of the present invention, and not as restrictive to a particular structure or method for implementing those principles.

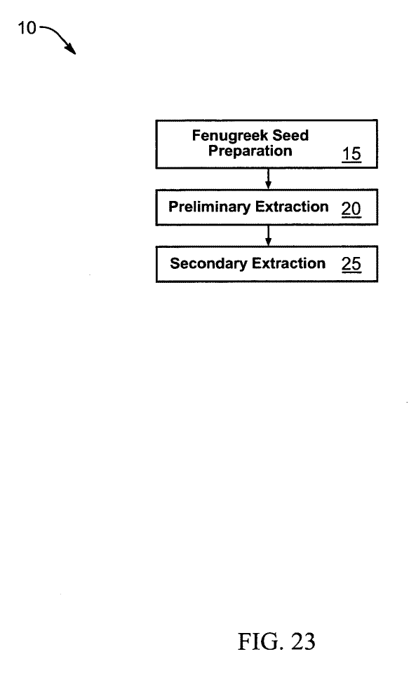

Turning now to FIG. 23 and consistent with the compositions described in Examples V-IX, an embodiment of a method of the present invention for extracting a novel composition of bio-active compounds (referred to herein as PROMILIN™) from fenugreek seeds may comprise the steps of: (1) providing a plurality of fenugreek seeds; (2) preparing the fenugreek seeds; and (3) extracting a composition of bio-active compounds from the prepared fenugreek seeds, wherein the composition preferably comprises 4-OH-Ile in an amount between about 60% and about 70% of a total weight of the amino acid content, together with one or more amino acids selected from the group consisting of glutamate in an amount between about 6% and about 8% of the total weight of the amino acid content, aspartate in an amount between about 4% and about 5% of the total weight of the amino acid content, arginine in an amount between about 2.4% and about 2.7% of the total weight of the amino acid content, cysteine in an amount between about 1% and about 2% of the total weight of the amino acid content, threonine in an amount between about 0.90% and about 1% of the total weight of the amino acid content, serine in an amount between about 4% and about 12% of the total weight of the amino acid content, glycine in an amount between about 2% and about 3% of the total weight of the amino acid content, alanine in an amount between about 3% and about 4% of the weight of the amino acid content, valine in an amount between about 1% and about 1.5% of the total weight of the amino acid content, methionine in an amount between about 0.35% and about 0.60% of the total weight of the amino acid content, isoleucine in an amount greater than 0.5% of the total weight of the amino acid content, and histidine in an amount between about 0.35% and about 0.40% of the total weight of the amino acid content (inclusive of any chemical salts, anhydrides, or isomers of any of the foregoing), in addition to, alkaloids, glycosides, volatile oils, saponins, sapogenins, mannans, flavonoids, fatty acids, vitamins and provitamins, minerals, and carbohydrates.

An embodiment of a method of the present invention for deriving, isolating, and/or extracting a composition of bio-active compounds (PROMILIN™) from fenugreek seeds may include the steps of: (1) soaking the fenugreek seeds in water and (2) crushing the fenugreek seeds. These steps of preparing the fenugreek seeds (i.e., soaking and crushing) are intended to separate the testa portion and the endosperm portion of the fenugreek seed. The additional steps of: (1) performing a preliminary extraction process and (2) performing a secondary extraction process are also contemplated and further disclosed herein.

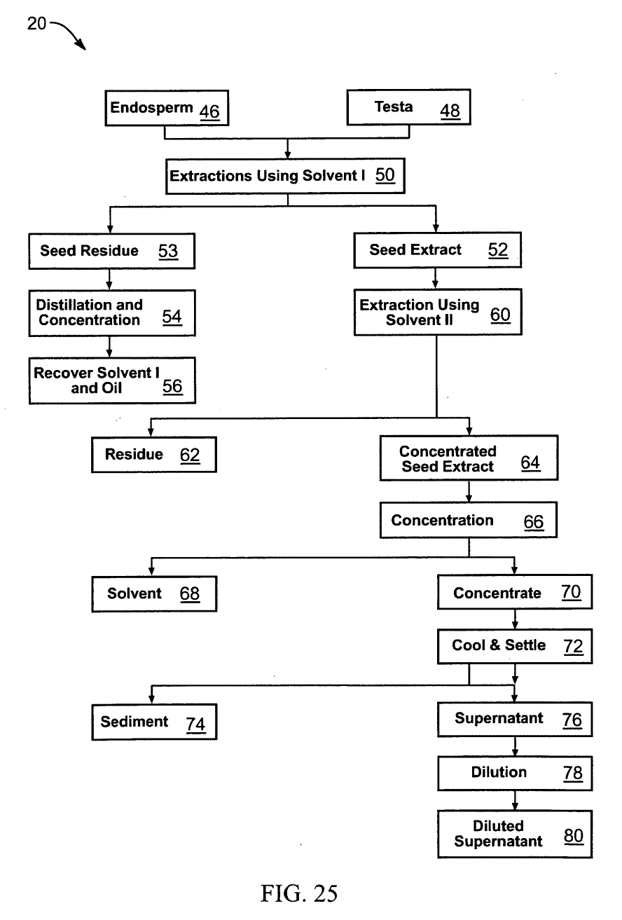

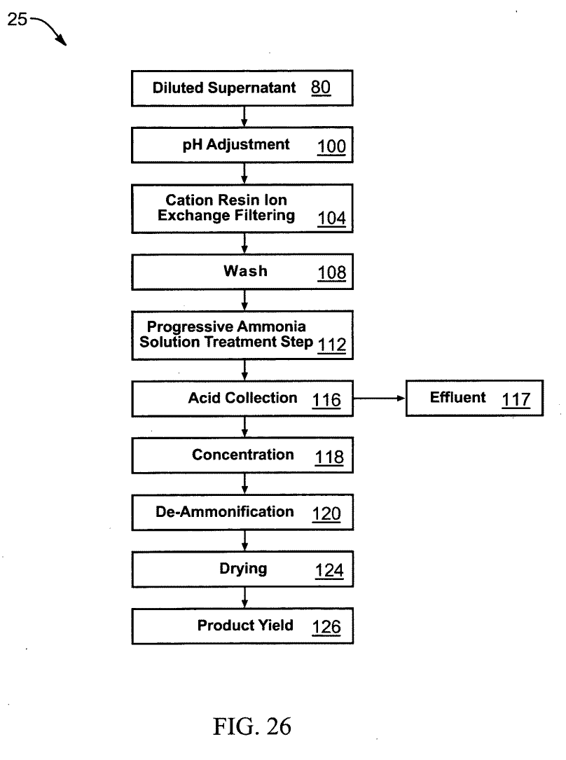

Referring to FIGS. 23-27, an embodiment of a method of the present invention for deriving, isolating, and/or extracting a composition of bio-active compounds (PROMILIN™) from fenugreek seeds, a preliminary extraction process may include the steps of: (1) performing one or more extractions on the prepared Fenugreek seeds using a first solvent at a temperature from between about 20° C. and about 90° C. and for a duration of between about one hour to about three hours to yield a seed residue and a seed extract; (2) distilling the seed residue using a fractionating column by heating the seed residue until boiling, capturing, and then cooling the heated vapors derived therefrom; (3) concentrating the distilled seed residue under vacuum to separate a Fenugreek seed oil and the first solvent; (4) performing one or more extractions of the seed extract using a second solvent at a temperature from between about 20° C. and about 90° C. and for a duration of between about one hour to about three hours to yield a second seed residue and a concentrated seed extract; (5) subjecting the concentrated seed extract to a further concentration under vacuum to separate a second concentrated seed extract from the second solvent; (6) cooling the second concentrated seed extract to room temperature; (7) settling of the second concentrated seed extract into crude protein and a supernatant; and (8) diluting the supernatant with de-ionized water to a volume between about two times and about ten times the volume of the supernatant.

In a further embodiment of a method of the present invention for deriving, isolating, and/or extracting a composition of bio-active compounds (PROMILIN™) from fenugreek seeds, a secondary extraction process may include the steps of: (1) adjusting the supernatant to a pH concentration from between about one and about 6.5 by diluting with an acid to produce a pH adjusted supernatant; (2) filtering the pH adjusted supernatant through a cation ion exchange resin to remove excess cations; (3) washing the cation ion exchange resin to remove contaminants from the resin-bound pH adjusted supernatant; (4) treating the resin_bound pH adjusted supernatant with an ammonia solution; (5) collecting a secondary extraction product acidic effluent and a non-acidic effluent from the cation ion exchange resin; (6) concentrating the acidic effluent under vacuum to separate contaminants; (7) removing residual ammonia solution from the secondary extraction product; and (8) drying the secondary extraction product to obtain 4-OH-Ile and one or more amino acids.

In yet another embodiment of a method of the present invention for deriving, isolating, and/or extracting a composition of bio-active compounds (PROMILIN™) from fenugreek seeds, a secondary extraction process may comprise the steps of: (1) filtering the supernatant through a cation ion exchange resin to remove excess cations; (2) washing the cation ion exchange resin to remove contaminants from the resin-bound supernatant; (3) treating the resin-bound supernatant with an ethanol treatment; (4) collecting a secondary extraction product acidic effluent; (5) adjusting the pH of the secondary extraction product acidic effluent from between about one and about 6.5 by diluting with an acid; (6) subjecting the pH adjusted secondary extraction product to a second filtration with a cation ion exchange resin; (7) treating the resin-bound pH adjusted secondary extraction product with an ammonia solution; (8) collecting a secondary extraction product acidic effluent and a non_acidic effluent; (9) concentrating the acidic effluent under vacuum to separate contaminants; (10) removing residual ammonia solution from the secondary extraction product; and (11) drying the secondary extraction product to obtain 4-OH-Ile and one or more amino acids.

Referring now to FIG. 23, an embodiment of a method for deriving, isolating, and/or extracting a composition of bio-active compounds (PROMILIN™), inclusive of 4-OH-Ile and one or more amino acids, from fenugreek seeds is illustrated. As shown, the method for deriving, isolating, and/or extracting a composition of bio-active compounds from fenugreek seeds 10 may include the steps of: (1) preparing the fenugreek seeds 15; (2) performing a preliminary extraction process 20; and (3) performing a secondary extraction process 25. Of course, the methods of deriving, isolating, and/or extracting a composition of bio-active compounds as taught by the present invention may include additional steps, as appreciated by those skilled in the art, in order to more optimally extract the useful bio-active compounds (e.g., 4-OH-Ile and one or more amino acids) from the fenugreek seeds.

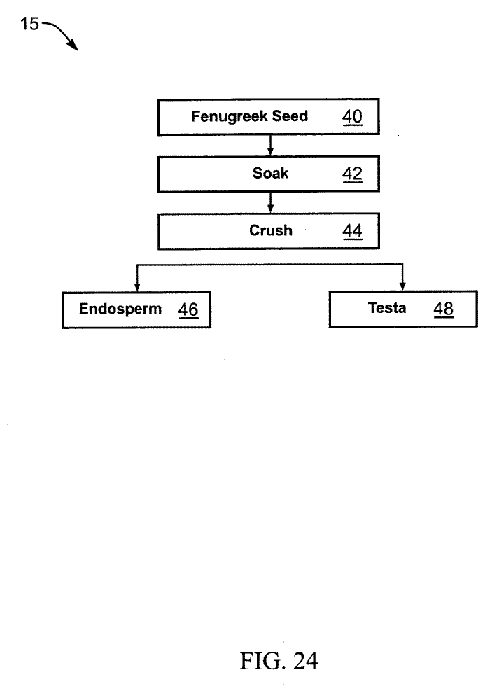

Referring now to FIGS. 23 and 24, one embodiment of the step for preparing the fenugreek seeds 15 of the present invention is shown comprising the steps of: (1) providing the fenugreek seeds 40; (2) soaking the fenugreek seeds 42; and (3) crushing the fenugreek seeds 44. The soaking step 42 preferably involves soaking the seeds in water for a specified amount of time. As appreciated, other solutions capable of providing the preparative properties of water may also be used. After the seeds have been soaked, the step of crushing the seeds 44 is intended to effectively separate various parts of the seed. For example, the crushing step 44 may separate the thick or hard outer coat of the seed, referred to as a testa 48, from the inner portion of the seed, known as the endosperm 46. As readily known to those skilled in the art, the endosperm 46 is a nutritive tissue that surrounds the plant embryo.

Referring specifically to FIG. 24, and generally to FIGS. 23 and 24, an embodiment of a step of performing a preliminary extraction process 20 from the endosperm 46 and the testa 48 resulting from the preparation steps 15 may include the steps of: (1) extracting 50 using a solvent (Solvent I). Solvent I may include, for example and not by way of limitation, a compound such as hexane, cyclohexane, ether, or any combinations thereof. The extraction step 50, as contemplated herein, effectively de-fats the fenugreek seeds. Accordingly, after performing the preliminary extraction process 20, the composition of bio-active compounds resulting from the process of the present invention may be referred to as “de-fatted.” The extraction step 50 may also involve repeatedly heating the combination of prepared fenugreek seeds and Solvent I.

For example, in an embodiment of the present invention, the combination of fenugreek seeds and Solvent I may be heated three times to temperatures ranging from between about 20° C. and about 90° C. If desired, the combination of seeds and Solvent I may be heated three times to temperatures ranging from between 65° C. and about 70° C. As appreciated, the combination of prepared fenugreek seeds and Solvent I may be maintained at these elevated temperatures for any of a range of time periods sufficient to achieve the desired results. In one embodiment of the present invention, the combination of prepared fenugreek seeds and Solvent I are maintained at elevated temperatures between about 1 hour and about 3 hours. Consequently, the extraction step 50 of the present invention typically yields a seed extract 52 and a seed residue 53.

Referring specifically now to FIG. 24, in an embodiment of the present invention a distillation and concentration step 54 may be performed on the fenugreek seed residue 53. As appreciated, the distillation and concentration step 54 may make use of a variety of conventional means to distill and concentrate extracts from the fenugreek seed. For example, distilling a seed residue obtained from successive extractions with a first solvent using a fractionating column may be accomplished by heating the seed residue until boiling, capturing, and then cooling the heated vapors. The distillation and concentration step 54 of the preliminary extraction step 20 of one embodiment of the present invention may yield quantities 56 of recovered Solvent I, as well as, fenugreek seed oil, diosgenin, fenugreek isoflavone, fenugreek saponin, and a soluble fiber, such as galactomannan or the like.

An extraction step 60 of an embodiment of a preliminary extraction step 20 of the present invention using a solvent (Solvent II) may be performed on the concentrated seed extract 52. Solvent II may comprise a solution including ethanol or a solvent having similar chemical properties to ethanol. The concentration of ethanol used in the extraction step 60 may assume a variety of values. For example, the ethanol concentration may vary between the values of between about 10% and about 95%.

In one embodiment, the extraction step 60 further involves the step of repeatedly heating the combination of fenugreek seed extract 52 and Solvent II. The combination may be heated three times to temperatures ranging between about 20° C. and about 90° C. Moreover, the combination of fenugreek seed extract 52 and Solvent II may be heated three times to temperatures ranging between about 65° C. and about 70° C. The combination of seed extract 52 and Solvent II may be maintained at these elevated temperatures for a broad range of time periods sufficient to achieve the desired results. For example, the combination of seed extract 52 and Solvent II may be maintained at elevated temperatures between about 1 hour and about 3 hours. Further to the process disclosed herein, one embodiment of the extraction step 60 of the present invention typically yields a seed residue 62 and a concentrated seed extract 64.

Additional steps associated with an embodiment of a preliminary extraction process 20 may include a concentration step 66 performed on the concentrated seed extract 64. The concentration step 66 preferably comprises the use of a vacuum to separate quantities of solvent 68 and a concentrate 70. The separated concentrate 70 may then be subject to a step of cooling and settling 72 to yield a sediment 74, including crude proteins, and a supernatant 76. A dilution step 78 may then be applied to the supernatant 76 to produce a diluted supernatant 80. As appreciated, the dilution step 78 may involve the addition of de-ionized water. The volume of water added may vary. For example, the amount of water added in the dilution step 78 of an embodiment of the present invention may include between about 2-10 times the volume of the supernatant 76. After dilution, the diluted supernatant 80 may then undergo a secondary extraction process 25, as described in FIGS. 23, 25, and 26.

As described herein, embodiments of a composition of bio-active compounds of the present invention (referred to as PROMILIN™ herein) include 4-OH-Ile and one or more compounds selected from the group consisting of amino acids, alkaloids, glycosides, volatile oils, saponins, sapogenins, mannans, flavonoids, fatty acids, vitamins and provitamins, minerals, and carbohydrates. Alkaloids, glycosides, volatile oils, saponins, sapogenins, mannans, flavonoids, fatty acids, vitamins and provitamins, minerals, and carbohydrates may be incorporated into embodiments of the present invention, as follows: Alkaloids may be selected from the group consisting of carpaine, gentianine, and trigonelline. Glycosides may be selected from the group consisting of 7-acetoxy-4-methylcoumarin, coumarin, luteolin, p-coumaric acid, rutin, trigofoenosides, trigonellosides, vitexin-2′-o-p-coumarate, yamogenin-3,26-biglycoside, and yamogenin-tetrosides. Volatile oils may be selected from the group consisting of 3-hydroxy-4,5-dimethyl-2-furanone, dihydrobenzofuran, dihydroactinidiolide, elemene, muurolene, and selinene. Saponins may be selected from the group consisting of 25-alpha-spirosta-3,5-diene and dioscin. Sapogenins may be selected from the group consisting of diosgenin, fenugreekine, gitogenin, neotigogenin, tigogenin, and yamogenin. Mannans may be selected from the group consisting of beta-mannan and galactomannan. Flavonoids may be selected from the group consisting of homoorientin, orientin, quercetin, trigoforin, trillin, vicenin-1, vicenin-2, vitexin, isovitexin, and luteolin. Fatty acids may be selected from the group consisting of arachidic acid, behenic acid, oleic acid, palmitic acid, and stearic acid. Vitamins and provitamins may be selected from the group consisting of acetylcholine, beta-carotene, choline, cyancobalamin, folacin, niacin, nicotinic acid, pyridoxine, riboflavin, thiamine, and xanthophyll. Minerals may be selected from the group consisting of calcium, chromium, cobalt, copper, iron, magnesium, manganese, phosphorous, potassium, selenium, silicon, sodium, sulfur, tin, and zinc. Carbohydrates may be selected from the group consisting of arabinose, d-mannose, raffinose, stachyose, and verbascose.

It will be readily understood by those of ordinary skill in the relevant art that the concepts and execution of all of the components of the present invention as described with reference to the Figures and Examples herein can be carried out and utilized in a variety of different configurations and using alternative methods yielding an essentially equivalent result, without departing from the essential characteristics of the invention. The scope of the invention is therefore defined solely by indicated by the appended claims. Thus, the present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A method for improving glycemic control in mammals, comprising the step of administering an amount of a composition comprising an amino acid content including 4-hydroxyisoleucine and glutamate, wherein said 4-hydroxyisoleucine comprises an amount between about 60% and about 70% of a total weight of said amino acid content and said cysteine comprises between about 1% and about 2% of the total weight of the amino acid content.

2. The method as defined in claim 1, wherein said mammal has a disorder in carbohydrate metabolism leading to a tendency towards excess blood sugar.

3. The method as defined in claim 1, wherein the composition comprises between about 10% and about 70% by weight of the 4-hydroxyisoleucine and chemical salts, anhydrides and isomers thereof.

4. The method as defined in claim 1, further comprising the step of administering between about 200 mg and about 400 mg of said composition.

5. The method as defined in claim 1, wherein said amino acid content is derived from seeds of fenugreek (Trigonella foenum graecum).

6. The method as defined in claim 1, wherein the step of administering the composition is performed shortly before eating.

7. The method as defined in claim 1, wherein said composition is administered in an oral form.

8. The method as defined in claim 1, wherein said administered composition comprises an amount selected to provide said 4-hydroxyisoleucine at between 0.05 mg/kg of body weight and 9 mg/kg of body weight of said mammal.

9. The method as defined in claim 1, wherein the composition comprises aspartate in an amount between about 4% and about 5% of the total weight of the amino acid content.

10. The method as defined in claim 1, wherein the composition further comprises one or more compounds selected from the group consisting of a glycoside, an alkaloid, a mannan, a flavonoid, a saponin, and a sapogenin.

11. A method of improving glucose tolerance in a mammal, comprising the step of administering an effective amount of a composition comprising an amino acid content including 4-hydroxyisoleucine, glutamate, and aspartate, wherein said 4-hydroxyisoleucine comprises an amount between about 60% and about 70% of a total weight of said amino acid content, the glutamate comprises an amount between about 6% and about 8% of the total weight of the amino acid content, and the aspartate comprises an amount between about 4% and about 5% of the total weight of the amino acid content.

12. The method as defined in claim 11, wherein said amino acid content comprises between about 20% and about 30% by weight of said 4-hydroxyisoleucine.

13. The method as defined in claim 11, wherein the amino acid content is derived from seeds of fenugreek (Trigonella foenum graecum).

14. The method as defined in claim 11, wherein said mammal has a disorder of carbohydrate metabolism tending to cause excess blood sugar levels.

15. The method as defined in claim 11, wherein the amino acid content further comprises one or more amino acids selected from the group consisting of arginine in an amount between about 2.4% and about 2.7% of the total weight of the amino acid content, cysteine in an amount between about 1% and about 2% of the total weight of the amino acid content, threonine in an amount between about 0.90% and about 1% of the total weight of the amino acid content, serine in an amount between about 4% and about 12% of the total weight of the amino acid content, glycine in an amount between about 2% and about 3% of the total weight of the amino acid content, alanine in an amount between about 3% and about 4% of the weight of the amino acid content, valine in an amount between about 1% and about 1.5% of the total weight of the amino acid content, methionine in an amount between about 0.35% and about 0.60% of the total weight of the amino acid content, isoleucine in an amount greater than 0.5% of the total weight of the amino acid content, and histidine in an amount between about 0.35% and about 0.40% of the total weight of the amino acid content.

16. The method as defined in claim 11, wherein the composition further comprises one or more compounds selected from the group consisting of a glycoside, an alkaloid, a mannan, a flavonoid, a saponin, and a sapogenin.

17. A method of improving glycemic control in humans having an insulin-resistant disorder of carbohydrate metabolism, comprising the step of administering a composition comprising an amino acid content including an amino acid content including 4-hydroxyisoleucine and glutamate, wherein said 4-hydroxyisoleucine comprises an amount between about 60% and about 70% of a total weight of said amino acid content and said glutamate comprises an amount between about 6% and about 8% of the total weight of the amino acid content.

18. The method as defined in claim 17, wherein said amino acid content is derived from seeds of fenugreek (Trigonella foenum graecum).

19. The method as defined in claim 17, wherein said composition comprises between about 20% and about 30% by weight of the 4-hydroxyisoleucine.

20. The method as defined in claim 17, wherein said step of administering said composition is performed shortly before a meal or snack.

21. The method as defined in claim 17, wherein said composition is administered in an oral form.

22. The method as defined in claim 17, wherein said amount of said composition comprises an amount of 4-hydroxyisoleucine at between 0.05 mg/kg of body weight and 9 mg/kg of body weight of said mammal.

23. The method as defined in claim 17, wherein said amino acid content further comprises one or more amino acid selected from the group consisting of aspartate in an amount between about 4% and about 5% of the total weight of the amino acid content, arginine in an amount between about 2.4% and about 2.7% of the total weight of the amino acid content, cysteine in an amount between about 1% and about 2% of the total weight of the amino acid content, threonine in an amount between about 0.90% and about 1% of the total weight of the amino acid content, serine in an amount between about 4% and about 12% of the total weight of the amino acid content, glycine in an amount between about 2% and about 3% of the total weight of the amino acid content, alanine in an amount between about 3% and about 4% of the weight of the amino acid content, valine in an amount between about 1% and about 1.5% of the total weight of the amino acid content, methionine in an amount between about 0.35% and about 0.60% of the total weight of the amino acid content, isoleucine in an amount greater than 0.5% of the total weight of the amino acid content, and histidine in an amount between about 0.35% and about 0.40% of the total weight of the amino acid content.
